SEQUENCE LISTING (16 sequences)

SEQ ID NO: 1 - protein, 101 aa, Homo sapiens
AHKLFIGGLPNYLNDDQVKELLTSFGPLKAFNLVKDSATGLSKGYAFCEYVDINVTDQAIAGLNGMQLGDKKLLVQRASVGAKNADYKDDDDKLEHHHHHH

SEQ ID NO: 2 - protein, 4 aa, Homo sapiens
NYLN

SEQ ID NO: 3 - protein, 7 aa, Homo sapiens
MQLGDKK

SEQ ID NO: 4 - protein, 4 aa, Artificial Sequence
  synthetic peptide C6 Loop 1
NGDN

SEQ ID NO: 5 - protein, 7 aa, Artificial Sequence
  synthetic peptide C6 Loop 2
GYPTDSC

SEQ ID NO: 6 - protein, 4 aa, Artificial Sequence
  synthetic peptide 1E2 Loop 1
VDYN

SEQ ID NO: 7 - protein, 7 aa, Artificial Sequence
  synthetic peptide 1H2 Loop 2
NVCHLHT

SEQ ID NO: 8 - protein, 4 aa, Artificial Sequence
  synthetic peptide 1H2 Loop 1
VDYN

SEQ ID NO: 9 - protein, 7 aa, Artificial Sequence
  synthetic peptide 1H2 Loop 2
SGSRIRA

SEQ ID NO: 10 - protein, 4 aa, Artificial Sequence
  synthetic peptide 3B5 Loop 1
ADGD

SEQ ID NO: 11 - protein, 7 aa, Artificial Sequence
  synthetic peptide 3B5 Loop 2
FATSRFS

SEQ ID NO: 12 - protein, 4 aa, Artificial Sequence
  synthetic peptide 4H5 Loop 1
DYYG

SEQ ID NO: 13 - protein, 7 aa, Artificial Sequence
  synthetic peptide 4H5 Loop 2
RFYGTAT

SEQ ID NO: 14 - protein, 128 aa, Artificial Sequence
  cAbBCII-10 VHH
QVQLVESGGGSVQAGGSLRLSCTASGGSEYSYSTFSLGWFRQAPGQEREAVAAIASMGGLTYYADSVKGRFTISRDNAKNTVTLQMNNLKPEDTAIYYCAAVRGYFMRLPSSHNFRYWGQGTQVTVSS

SEQ ID NO: 15 - protein, 16 aa, Artificial Sequence
  CDR3 of 12G mutant
PPYQHDHYIFYNTNDS

SEQ ID NO: 16 - protein, 16 aa, Artificial Sequence
  CDR3 of 738 mutant
TNHSNEQTANHDNYIH

(19) United States
(12) Patent Application Publication
(10) Pub. No.: US 2025/0182853 A1
(43) Pub. Date: Jun. 5, 2025

(54) METHOD FOR PRODUCING LIBRARY BY MACHINE LEARNING

(71) Applicants: TOHOKU UNIVERSITY, Sendai-shi (JP); NATIONAL INSTITUTE OF ADVANCED INDUSTRIAL SCIENCE AND TECHNOLOGY, Tokyo (JP)

(72) Inventors: Mitsuo UMETSU, Miyagi (JP); Hikaru NAKAZAWA, Miyagi (JP); Tomoyuki ITO, Miyagi (JP); Sakiya KAWADA, Miyagi (JP); Hafumi NISHI, Miyagi (JP); Tomoshi KAMEDA, Tokyo (JP); Yutaka SAITO, Tokyo (JP); Thuy Duong NGUYEN, Tokyo (JP); Yoichi KURUMIDA, Tokyo (JP); Koji TSUDA, Tokyo (JP)

(73) Assignees: TOHOKU UNIVERSITY, Sendai-shi (JP); NATIONAL INSTITUTE OF ADVANCED INDUSTRIAL SCIENCE AND TECHNOLOGY, Tokyo (JP)

(21) Appl. No.: 18/840,105

(22) PCT Filed: Mar. 10, 2022
(86) PCT No.: PCT/JP2022/010438

§ 371 Date: Aug. 21, 2024

(51) Int. Cl. G16B 35/10; G16B 35/20; G16B 40/00

(52) U.S. Cl. CPC G16B 35/10; G16B 35/20; G16B 40/00

(57) ABSTRACT

A method for producing a nucleic acid library. The method includes: preparing, by a phage display method, a first library composed of mutants obtained by randomly introducing a mutation into a nucleic acid sequence encoding a protein bound to or configured to be bound to a target; performing biopanning on the first library and obtaining data to be used for machine learning from an obtained sublibrary; and performing machine learning using the data and obtaining a second library from the first library based on machine learning prediction. The data to be used for machine learning includes a sequence of a mutant population included in a sublibrary at a target-binding sequence elution stage, an estimated binding strength to the target, and an actual measurement value of binding of some mutants included in the mutant population to the target.

### TECHNICAL FIELD

The present invention relates to a method for producing a nucleic acid library by machine learning. More specifically, the present invention relates to a method for producing, by using more appropriate data as machine learning data, a nucleic acid library containing many nucleic acids encoding a desired protein.

### BACKGROUND ART

There is a wide need for modifying a functional protein such as an antibody or an enzyme to improve functions of the functional protein. Recently, studies for more efficiently modifying the functions of proteins by using machine learning have been advanced. In these studies, a mutant library is produced on a certain scale, amino acid sequences and functions of mutants are experimentally measured, and the associated data is used as training data for constructing a machine learning model for predicting a function based on a sequence. Then, the constructed machine learning model is used to predict a mutant whose functions are predicted to be improved.

Regarding a data set of machine learning, two types of data sets that directly or indirectly associate an amino acid sequence with function and physical property values are applied. In the direct association data set, the function and physical property values of each mutant are measured for each mutant, and these function and physical property values are associated with a sequence of the corresponding mutant (NPL 1, etc.). On the other hand, in the indirect association data set, the function and physical property values are not directly measured, and a data set is created by using the number of reads of amino acid sequences obtained by deep sequence analysis as a substitute for the function and physical property values (NPLs 2 and 3).

The direct association between an amino acid sequence and a function and physical property value may be a high-quality data set for machine learning, and it is difficult to create a large-scale data set. The size is limited to several tens to several hundreds of sizes, and a searchable sequence is also limited. On the other hand, the quality of the indirect association data is lower than that of the direct association data set, and large-size amino acid sequence data that can be acquired by the deep sequence analysis can be used. Therefore, when the location and the number of mutation residues and the amino acids that appear are limited, the direct association data set is often applied, and the indirect association data set is often applied to the discovering of antibody lead molecules using a molecular display methods.

Biopanning from a molecular library using a phage display method (see FIG. 1A) is an effective method for obtaining antibody fragments and antibody-like molecules showing target-binding from a large-scale mutant group of about 10¹⁰. In recent years, an operation has been reported in which machine learning is performed by estimating sequences having a high abundance rate (high enrichment rate) in a library after a selection operation as sequences with high binding force based on the results of sequence analysis using next generation sequencer (NGS) (PTL 1). In the past, machine learning is performed using data of a population after E. coli infection ((v) in FIG. 1A) or after phage amplification ((vi) in FIG. 1A) in the phage display method (NPL 2). However, in reality, it is often impossible to obtain a phage population containing mutants with significantly improved functions (target-binding property). Bias selection in which the enrichment rate changes depending on infection of E. coli with phages and the amplification process in addition to the target-binding property is present, and therefore, sequences with higher enrichment rate do not necessarily have an improved desirable function (NPL 4).

When a certain high-level proposed sequence is produced based on the result of machine learning prediction, it is necessary to synthesize each gene of the sequence from the sequence diversity, and the number of sequences to be evaluated is limited in terms of cost. Therefore, a sequence with the desired function cannot be obtained depending on the accuracy of the training data. Therefore, in a method according to the related art, the scale of the second library is small.

### CITATION LIST

### Patent Literature

PTL 1: US2019/0065677

### Non Patent Literature

NPL 1: Saito et al., “Machine-Learning-Guided Mutagenesis for Directed Evolution of Fluorescent Proteins” ACS Synth Biol. 2018; 7 (9): 2014-2022
NPL 2: Liu et al., “Antibody complementarity determining region design using high-capacity machine learning” Bioinformatics, 2020; 36 (7): 2126-2133
NPL 3: Saka et al., “Antibody design using LSTM based deep generative model from phage display library for affinity maturation” Scientific Reports, 2021; 11 (1): 5852
NPL 4: Ito et al., “Application of next-generation sequencing analysis in the directed evolution for creating antibody mimic” 65th Annual Meeting of the Biophysical Society. 2021.2.25. (Boston, MA, USA)

### SUMMARY OF INVENTION

### Technical Problem

An object of the present invention is to provide a library containing a nucleic acid encoding a desired protein. In particular, the present invention provides a method for obtaining a library containing a desired functional molecule even from a biopanning operation by which a clear positive mutant is not obtained.

### Solution to Problem

The estimated binding strength to the target was calculated using sequence data of sublibraries at various stages, and the correlation with the actual measurement value of the mutant was evaluated. Then, it was found that, by using the data of the sublibrary at the target-binding sequence elution stage ((iv) in FIG. 1A), the estimated binding strength having a high correlation with the actual measurement value can be obtained even when the enrichment of the sequence due to the selective pressure caused by the target-binding than the enrichment of the sequence caused by the bias selection in the infection of the E. coli with the phage and the amplification process. Further, the present inventors have found that degenerate codon design is combined with a sequence population predicted by machine learning from the indirect association data set and a secondary library also containing sequences similar to the sequences predicted by machine learning is constructed, so that libraries containing more desired proteins can be constructed at low cost.

That is, the present invention relates to the following [1] to [11].

[1] A method for producing a nucleic acid library, the method including:
1) a step of preparing, by a phage display method, a first library composed of mutants obtained by randomly introducing a mutation into a nucleic acid sequence encoding a protein bound to or to be bound to a target;
2) a step of performing biopanning on the first library and obtaining data to be used for machine learning from an obtained sublibrary; and
3) a step of performing machine learning using the data and obtaining a second library from the first library based on machine learning prediction, wherein the data to be used for machine learning includes a sequence of a mutant population included in a sublibrary at a target-binding sequence elution stage, an estimated binding strength to the target, and an actual measurement value of binding of some mutants included in the mutant population to the target.

[2] The method according to [1], in which the data to be used for machine learning is obtained by the following steps
i) obtaining data of sequences and appearance frequencies of the sequences for the sublibrary at the target-binding sequence elution stage and sublibraries at one or two or more stages different from the stage,
ii) calculating, based on the appearance frequencies, a score indicating the estimated binding strength to the target, and
iii) determining, as the data to be used for machine learning, the score, the actual measurement value of binding to the target, and sequence data providing the score and the actual measurement value.

[3] The method according to [2], in which
the different one or two or more stages are stages selected from the group consisting of a non-specific binding sequence removal stage, a target-binding sequence selection stage, an E. coli infecting operation stage, and a selected sequence amplification stage in a same round, stages selected from the group consisting of a non-specific binding sequence removal stage, a target-binding sequence selection stage, a target-binding sequence elution stage, an E. coli infecting operation stage, and a selected sequence amplification stage in different rounds, or both of them.

[4] The method according to [2], in which
the score is calculated using a ratio of an appearance frequency between the sublibrary at the target-binding sequence elution stage and a sublibrary at a non-specific binding sequence removal stage or a selected sequence amplification stage.

[5] The method according to [2], in which
the score is calculated by using a ratio of an appearance frequency in the sublibrary at the target-binding sequence elution stage to an appearance frequency in a sublibrary at a non-specific binding sequence removal stage in the same round, or calculated by using a ratio of an appearance frequency in the sublibrary at the target-binding sequence elution stage to an appearance frequency in a sublibrary at a selected sequence amplification stage in different rounds.

[6] The method according to [2], in which
the score is calculated using data of sublibraries at the 2nd to 4th round.

[7] The method according to [2], in which
the score is calculated according to any one selected from the following formulas 1) to 6):

$$[ Math . 1 ]$$

$${f}_{x} ( i ) = \frac{{F}_{x , 4} ( i )}{{F}_{x , 2} ( i )} \qquad 1 )$$

$${f}_{x} ( i ) = \frac{{F}_{x - 1 , 4} ( i )}{{F}_{x - 1 , 2} ( i )} \times \frac{{F}_{x , 4} ( i )}{{F}_{x , 2} ( i )} \qquad 2 )$$

$${f}_{x} ( i ) = \frac{{F}_{x - 1 , 4} ( i )}{{F}_{x - 1 , 2} ( i )} \times \frac{{F}_{x , 4} ( i )}{{F}_{x , 2} ( i )} \times \frac{{F}_{x + 1 , 4} ( i )}{{F}_{x + 1 , 2} ( i )} \qquad 3 )$$

$${f}_{x} ( i ) = \frac{{F}_{x , 4} ( i )}{{F}_{x - 1 , 6} ( i )} \qquad 4 )$$

$${f}_{x} ( i ) = \frac{{F}_{x - 1 , 4} ( i )}{{F}_{x - 2 , 6} ( i )} \times \frac{{F}_{x , 4} ( i )}{{F}_{x - 1 , 6} ( i )} \qquad 5 )$$

$${f}_{x} ( i ) = \frac{{F}_{x - 1 , 4} ( i )}{{F}_{x - 2 , 6} ( i )} \times \frac{{F}_{x , 4} ( i )}{{F}_{x - 1 , 6} ( i )} \times \frac{{F}_{x + 1 , 4} ( i )}{{F}_{x , 6} ( i )} \qquad 6 )$$

in which F_{x,n}(i) represents an abundance rate of a mutant i in the x-th round in a sublibrary n (number of reads of unique sequence/total number of reads of sublibrary), and n is as follows:
n=1: first library
n=2: sublibrary from phages removed by non-specific binding phage removal
n=3: sublibrary from phages removed at target-binding sequence elution stage
n=4: sublibrary from phages after target-binding
n=5: sublibrary from E. coli after being infected with phages
n=6: sublibrary from phages after amplification.

[8] The method according to any one of [1] to [7], in which
the actual measurement value of binding to the target is a value measured by ELISA.

[9] The method according to any one of [1] to [8], in which
in the step 3, the second library includes a sequence not predicted by machine learning, depending on design of a degenerate codon.

[10] The method according to any one of [1] to [9], in which
the protein bound to or to be bound to the target is an antibody, an antibody-like molecule, or an enzyme.

[11] A method for producing an optimized protein, the method including:
a step of obtaining a second library according to the method according to any one of claims 1 to 10;
a step of screening the second library to determine a nucleic acid sequence encoding an optimized protein; and
a step of producing a protein optimized based on the nucleic acid sequence.

### Advantageous Effects of Invention

The present invention has the following features: (1) using a sublibrary at a target-binding sequence elution stage as a phage population at an appropriate stage; (2) producing a second library for a space including more sequences rather than including only a top sequence predicted by machine learning; and (3) using a phage display method again to implement the second library at low cost.

According to the present invention, a library including more nucleic acids encoding a desired protein can be constructed. Accordingly, it is possible to efficiently improve the function of an industrially useful protein such as an antibody or an enzyme.

### BRIEF DESCRIPTION OF DRAWINGS

FIG. 1A shows an example of biopanning. FIG. 1B shows biopanning in Embodiments 1 and 2.

FIG. 2 shows an amino acid sequence of 2u2f protein.

FIG. 3 shows polyclonal phage ELISA using amplified phages after each round. The binding of the amplified phages was evaluated using the diluted phages from an amount of 5.0×10¹¹ cfu to undilution, 5-fold dilution, and 25-fold dilution. Each sample is detected by an anti-M13 phage-HRP antibody.

FIG. 4 shows evaluation of physical properties and functions of a C6 mutant. (A) of FIG. 4 shows purification of the C6 mutant performed by size exclusion chromatography (arrow indicates monomer fraction). (B) of FIG. 4 shows binding evaluation of the C6 mutant performed by ELISA. (Black): binding signals in wells with immobilized Galectin-3 via NeutrAvidin (Gray): binding signals in wells only with immobilized NeutrAvidin (no Galectin-3). (C) of FIG. 4 shows a CD spectrum measurement of the C6 mutant (gray) and wild type 2u2f (black).

FIG. 5 shows a ratio of the number of reads of a unique sequence in each sublibrary.

FIG. 6 shows a change in abundance rates between sublibraries for each unique sequence. An oblique straight line in the drawing indicates a reference line of y=x. Each axis indicates a logarithmic value of the abundance rate of a mutant in a sublibrary of interest. (A): Changes in abundance rates of amplified phages from 1st round to 2nd round (left part), from 2nd round to 3rd round (middle part), from 3rd round to 4th round (right part)

(B): Changes in abundance rates from input (amplified phages in the previous round) to output (eluted phages) in 2nd round (left part), 3rd round (middle part), and 4th round (right part)

FIG. 7 shows score value calculation. F_{x, n}: abundance rate in a sublibrary n at the x-th round (the number of reads of unique sequence/the total number of reads of the sublibrary)

FIG. 8 shows changes in appearance frequencies of amino acids at each residue location in 2nd and 3rd rounds. Amino acid appearance frequency (−1.0-1.0)=log₂ (amino acid appearance frequency of eluted phages (2nd)/amino acid appearance frequency of amplified phages (1st))

FIG. 9 shows appearance frequencies of amino acids at each residue location of top 10,000 sequences predicted by machine learning.

FIG. 10 shows clustering results of the top 10,000 sequences predicted by machine learning. (A) of FIG. 10 shows the number of sequences and an amino acid appearance frequency in each cluster. (B) of FIG. 10 shows a rank distribution of sequences included in each cluster (arrow: a cluster containing top 1000 sequences).

FIG. 11 shows appearance frequencies of amino acids at each residue location of a designed library (left: sequence predicted by machine learning, right: designed library).

FIG. 12 shows polyclonal phage ELISA using amplified phages after each round. From left to right in each graph, amounts of the amplified phages are respectively 5.0×10¹¹ cfu, 1.0×10¹¹ cfu, 2.0×10¹⁰ cfu (Target: Gal-3 (+)), 5.0×10¹¹ cfu, 1.0×10¹¹ cfu, 2.0×10¹⁰ cfu (Target: Gal-3 (−)) (Gal-3 (+)): binding signals in wells with immobilized Galectin-3 via NeutrAvidin (Gal-3 (−)): binding signals in wells only with immobilized NeutrAvidin (without Galectin-3)

FIG. 13 shows binding evaluation of 12 kinds of promising mutants using ELISA. (Gal-3 (+)): binding signals in wells with immobilized Galectin-3 via NeutrAvidin (Gal-3 (−)): binding signals in wells only with immobilized NeutrAvidin (without Galectin-3)

FIG. 14 shows EC₅₀ measurement results of 1E2, 1H2, 3B5, and 4H5 mutants.

FIG. 15 shows a CD spectrum measurement of wild-type 2u2f, 1H2, 1E2, 3B5, and 4H5.

FIG. 16 shows an amino acid sequence of cAbBCII-10 and mutation sites (frame: CDR defined by AbM).

FIG. 17 shows results of polyclonal phage ELISA. From left to right in each graph, the amounts are respectively 5.0×10¹⁰ cfu, 1.7×10¹⁰ cfu, 5.6×10⁹ cfu, 1.9×10⁹, 6.2×10⁸ cfu, 2.1×10⁸ cfu, and 6.9×10⁷ cfu. (A): binding signals in wells with immobilized Galectin-3 via NeutrAvidin (B): binding signals in wells only with immobilized NeutrAvidin (without Galectin-3)

[FIG. 18] (A) of FIG. 18 shows SEC results of a wild-type VHH (upper part) and a 12G mutant (lower part), in which an arrow indicates a monomer. (B) of FIG. 18 shows ELISA results (black: target molecule is present, gray: no target molecule). (C) of FIG. 18 shows CD spectrum results (black: wild-type VHH, gray: 12G).

FIG. 19 shows changes in a mutant group distribution during an in-vitro selection operation process (left end: initial phage, from left to right in each round: negative phage, washed phage, eluted phage, infected E. coli, amplified phage).

[FIG. 20] (A) of FIG. 20 shows SEC results of a wild-type VHH (upper part) and a 738 mutant (lower part), in which an arrow indicates a monomer. (B) of FIG. 20 shows ELISA results (black: target molecule is present, gray: no target molecule). (C) of FIG. 20 shows CD spectrum results (black: wild-type VHH, gray: 738).

[FIG. 21] (A) of FIG. 21 shows SEC results of 2G and 6C mutants. (B) of FIG. 21 shows CD spectrum results (from upper part to lower part: WT, 738, 6C, and 2G).

[FIG. 22] (A), (B), and (C) of FIG. 22 show ELISA results of 2G and 6C mutants, in which (A): binding signals in wells with immobilized Galectin-3 via NeutrAvidin, (B): binding signals in wells only with immobilized NeutrAvidin (without Galectin-3), and (C): binding signals in wells with immobilized BSA (without Galectin-3). (D) of FIG. 22 shows ELISA results obtained by changing concentrations of 2G and 6C mutants with respect to wells with immobilized Galectin-3.

### DESCRIPTION OF EMBODIMENTS

The present t invention relates to a method for producing a nucleic acid library by a phage display method.

### 1. Production of Initial Library (First Library)

First, a library composed of mutants obtained by randomly introducing mutations into a protein that is “bound to or to be bound to a target” is prepared according to the phage display method. In this specification, the library prepared at first is referred to as an “initial library” or a “first library” in order to distinguish the library from a library after enrichment by machine learning. The “initial library” and the “first library” are used interchangeably in this specification.

The “protein bound to or to be bound to a target” is not particularly limited, and a functional protein requiring improvement in properties, such as an antibody, an antibody-like molecule, or an enzyme, is preferred. Examples of the antibody include low-molecular antibodies such as a VHH antibody, and antibody fragments such as Fab, F(ab′)², scFv, a diabody, and a minibody. The antibody-like molecule exhibits a function by specifically binding to an antigen as in the case of an antibody, and means a compound structurally unrelated to antibodies and is also referred to as an antibody mimetic. Examples of the antibody-like molecule include an affibody, an affimer, affitin, an alphabody, an anticalin, an avimer, a phynomer, a monobody, DARPins, and a nanoCLAMP.

As a site into which a mutation is introduced (“mutation site”), a site that affects properties to be optimized is selected. The expression “affecting the properties” means that the properties are changed or improved by changes (substitutions, deletions, insertions) of an amino acid at the site, particularly by the amino acid substitution.

For example, in the case of an antibody, selection of a mutation site is selection of a residue including a complementarity-determining region (CDR) which is an antigen recognition site and a periphery thereof, and CDR is defined by Chothia, AbM, Kabat, Contact, or the like. Regarding an antibody-like molecule of a non-antibody protein, a reported mutation introduction site can be selected, and the mutation site can also be selected based on a degree of exposure to a surface and an appearance frequency of an amino acid at each residue location in a homologous protein present in nature.

When a selective pressure for improving the structural stability is applied without impairing a binding function, the selection of the mutation site can be performed based on consensus engineering. The term “consensus engineering” refers to a design based on a consensus (consensus design or consensus-based engineering), and is an approach for enhancing the stability of proteins by modifying a sequence of a protein so as to be close to a consensus sequence obtained from alignment of a large number of proteins from a specific family (Porebski and Buckle, “Consensus protein design” Protein Engineering, Design & Selection, 2016, 29 (7): 245-251, Steipe B., et al., J. Mol. Biol, 1994, 240 (3): 188-192, etc.).

Specifically, in the case of functional modification of an enzyme (improvement in thermal stability of an enzyme or the like), based on the assumption that a large number of amino acid residues selected in nature contribute to the improvement in the function of the enzyme, an amino acid sequence group of proteins belonging to the same family as an amino acid sequence of a starting protein is subjected to a multiple sequence alignment method (ClustalW, MAFFT, or the like) to calculate an appearance frequency of an amino acid at each residue location, and the amino acid residue stored at the highest frequency is defined as a consensus residue. Then, a location of each amino acid residue of the starting protein is mutated to the consensus residue. On the other hand, regarding antibodies, based on the assumption that various sudden mutations observed in a germ cell line family result from the elimination of a sudden mutation causing structural destabilization, an amino acid most frequently observed at a specific location of alignment of a variable region fragment of an immunoglobulin (Ig) is considered to be the most preferred amino acid in terms of thermodynamic stability.

By using the consensus engineering, the functional modification of a protein can be carried out only by an amino acid sequence without requiring knowledge of crystal structures or complicated in-silico calculation. However, when an amino acid that does not use a consensus residue is simply substituted with a consensus residue, the structural stability is adversely reduced, or other functions (for example, enzyme activity and antigen binding activity) are often reduced even if the structural stability is improved. Therefore, it is important to select the corresponding residue location and an amino acid caused to appear at the location.

Introduction of a mutation may be performed by methods known in the field, such as an overlap extension PCR method using a primer having a degenerate codon, an error-prone PCR method, a random primer method, an inverse PCR method, DNA shuffling, a staggered PCR method, a Kunkel method, and a quick change method. A commercially available mutation introduction kit can also be used.

A size of the library is not particularly limited, and is appropriately determined according to the number of mutation introduction sites. There are 20 kinds of natural amino acids, and therefore, for example, when the mutation introduction site has 3 residues, the size is about 8000 in 203, and when the mutation introduction site has 4 residues, the size is about 160000 in 20⁴. The method according to the present invention can be suitably used particularly when the function of binding to a target is changed, and when the mutation introduction site has 7 or more residues.

### 2. Acquisition of Machine Learning Data

Next, biopanning is performed on the first library, and data used for machine learning is acquired from the obtained sublibrary.

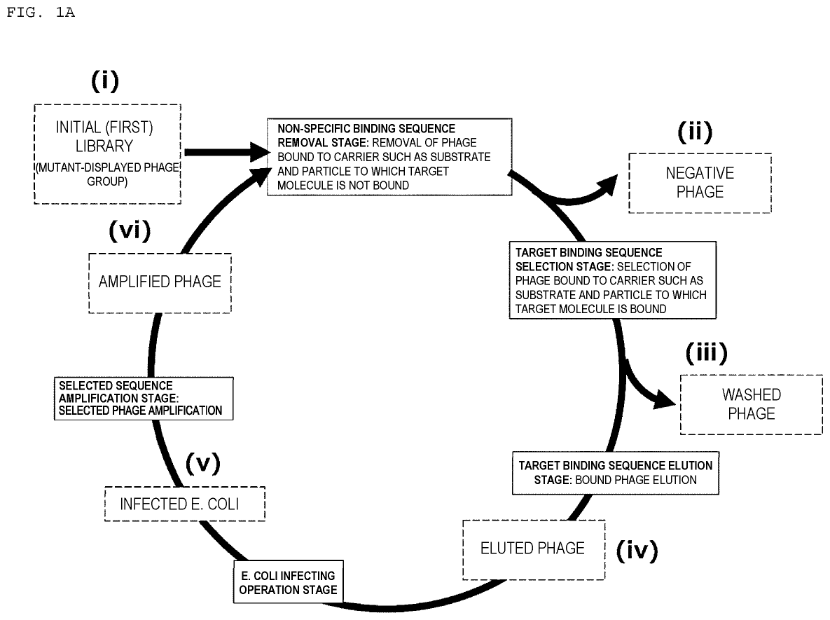

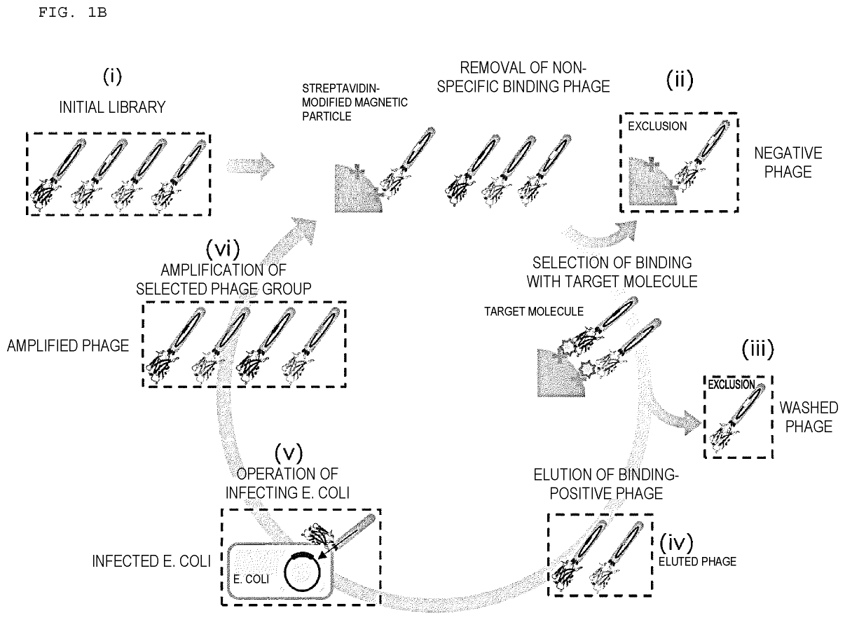

The term “biopanning” refers to an operation of enriching a target protein based on selection using specific binding to a target (see FIG. 1A). For example, when the target protein is an antibody or an antibody-like molecule, the biopanning is performed for binding to an antigen, and in the case of an enzyme, the biopanning is performed for binding to a substrate.

In a population included in a library, it is assumed that a sequence whose abundance rate (high enrichment rate) in the library is increased by biopanning has a strong binding force to a target. Therefore, regarding the mutant population (sublibrary) included in each stage of biopanning, sequences (amino acid sequences and nucleic acid sequences) and appearance frequencies thereof (the number of reads of a certain mutant/the total number of reads in the sublibrary) are analyzed to determine an enrichment rate of each sequence, and the enrichment rate is defined as an “estimated binding strength” to the target. The “estimated binding strength” is scored for use in machine learning.

As described above, in a method according to the related art, data (enrichment rate) of a population after E. coli is infected with a selected phage ((v) in FIG. 1A) or after the phage is amplified ((vi) in FIG. 1A) is used for machine learning. However, a bias is applied to an appearance frequency of a population after E. coli infection or phage amplification, and actual measurement values are not reflected. The inventors have analyzed sequences and appearance frequencies of mutant populations included in sublibraries at various stages of biopanning, scored the estimated binding strengths by various calculation formulas, and compared the correlations with the actual measurement values. As a result, the inventors have found that the data of the population after the target-binding sequence elution (iv) has a high correlation with the actual measurement value. In the biopanning, it is common that the enrichment rate after the target-binding sequence elution is lower than the enrichment rate of the population after the E. coli infection or the phage amplification. In this case, target-binding enrichment rate is obscured by bias changes that occur during the E. coli infection or the phage amplification, and enrichment by a selection operation is not observed.

The “stage” of biopanning is, for example, a non-specific binding sequence removal stage, a target-binding sequence selection stage, a target-binding sequence elution stage, an E. coli infecting operation stage, and a selected sequence amplification stage in each round of biopanning.

The data used for the machine learning in the present invention includes a sequence of a mutant population included in a sublibrary at the target-binding sequence elution stage, an estimated binding strength to a target, and an actual measurement value of binding to the target.

The data used for the machine learning is acquired by, for example, the following steps:
i) a step of obtaining, for a target-binding sequence elution stage ((iv) in FIG. 1A) in biopanning or one or two or more stages different from the above stage, data of a sequence of a mutant population included in each stage and an appearance frequency of the sequence;
ii) a step of calculating a score indicating an estimated binding strength to a target from the appearance frequency (for example, normalizing the score to a numerical value of 0 to 1); and
iii) a step of determining, as data used for machine learning, the score, an actual measurement value of binding to the target, and sequence data providing the score and the actual measurement value.

The number of sequences analyzed in mutants in each sublibrary is not particularly limited as long as training data that is meaningful in artificial intelligence can be provided. The number of sequences (for example, 10⁹ sequences) in the initial library subjected to the selection operation is preferred, and the number of sequences may be 100,000 or more.

In the present invention, the number of rounds of biopanning is not particularly limited, and is appropriately set depending on the number of mutants as objects and the affinity with a target. In general, the biopanning is performed for two or more rounds, preferably three or more rounds or four or more rounds, generally two rounds to six rounds, and particularly two rounds to four rounds.

The different one or two or more stages may be stages different from the target-binding sequence elution stage in the same round, stages in different rounds, or both of them. The different one or two or more stages are preferably one or two or more stages different from the target-binding sequence elution stage in the same round.

Specifically, examples of one or two or more different stages include a stage selected from the group consisting of a non-specific binding sequence removal stage, a target-binding sequence selection stage, an E. coli infecting operation stage, and a selected sequence amplification stage in the same a stage selected from the group consisting of a non-specific binding sequence removal stage, a target-binding sequence selection stage, a target-binding sequence elution stage, an E. coli infecting operation stage, and a selected sequence amplification stage in different rounds, and both of them. As one or two or more different stages, the non-specific binding sequence removal stage and/or the selected sequence amplification stage are/is preferred, and the non-specific binding sequence removal stage is more preferred.

The score is, for example, a normalized and standardized numerical value calculated by using a ratio of an appearance frequency in the sublibrary at the target-binding sequence elution stage to an appearance frequency in the sublibrary at the non-specific binding sequence removal stage or the selected sequence amplification stage. More specifically, the score is a normalized and standardized numerical value calculated by using a ratio of an appearance frequency in the sublibrary at the target-binding sequence elution stage to an appearance frequency in the sublibrary at the non-specific binding sequence removal stage in the same round, or calculated by using a ratio of an appearance frequency in the sublibrary at the target-binding sequence elution stage to an appearance frequency in the sublibrary at the selected sequence amplification stage in different rounds.

The score is calculated by using data of the sublibraries at the 2nd round, the 3rd round, the 4th round, or the 5th round, preferably the 2nd round to the 4th round.

The score is calculated based on, for example, any one of the following formulas 1) to 6).

$$[ Math . 2 ]$$

$${f}_{x} ( i ) = \frac{{F}_{x , 4} ( i )}{{F}_{x , 2} ( i )} \qquad 1 )$$

$${f}_{x} ( i ) = \frac{{F}_{x - 1 , 4} ( i )}{{F}_{x - 1 , 2} ( i )} \times \frac{{F}_{x , 4} ( i )}{{F}_{x , 2} ( i )} \qquad 2 )$$

$${f}_{x} ( i ) = \frac{{F}_{x - 1 , 4} ( i )}{{F}_{x - 1 , 2} ( i )} \times \frac{{F}_{x , 4} ( i )}{{F}_{x , 2} ( i )} \times \frac{{F}_{x + 1 , 4} ( i )}{{F}_{x + 1 , 2} ( i )} \qquad 3 )$$

$${f}_{x} ( i ) = \frac{{F}_{x , 4} ( i )}{{F}_{x - 1 , 6} ( i )} \qquad 4 )$$

$${f}_{x} ( i ) = \frac{{F}_{x - 1 , 4} ( i )}{{F}_{x - 2 , 6} ( i )} \times \frac{{F}_{x , 4} ( i )}{{F}_{x - 1 , 6} ( i )} \qquad 5 )$$

$${f}_{x} ( i ) = \frac{{F}_{x - 1 , 4} ( i )}{{F}_{x - 2 , 6} ( i )} \times \frac{{F}_{x , 4} ( i )}{{F}_{x - 1 , 6} ( i )} \times \frac{{F}_{x + 1 , 4} ( i )}{{F}_{x , 6} ( i )} \qquad 6 )$$

In the formula, F_{x, n} (i) represents an abundance rate (the number of reads of a unique sequence/the total number of reads of a sublibrary) of a mutant i in the x-th round in a sublibrary n.
n is as follows.
n=1: initial library (first library)
n=2: sublibrary from phages removed by non-specific binding phage removal
n=3: sublibrary from phages removed at target-binding sequence elution stage
n=4: sublibrary from phages after target-binding
n=5: sublibrary from E. coli after being infected with phages
n=6: sublibrary from phages after amplification

Which function is to be selected as the function fx (i) can be determined according to an AUC (Area Under Curve) value obtained by calculating a numerical value associated with a sequence using each function. For example, an appropriate function can be selected from functions that give an AUC value of 0.5 or more, 0.6 or more, or 0.7 or more.

The score may be further normalized as necessary. For example, as in Examples 1 and 2 to be described below, a logarithm of a value of the “estimated binding strength” is defined as an enrichment rate (ER (i)), and nScore (i) is determined in order to normalize the score with a larger ER (i) value as being better.

$$[ Math . 3 ]$$

$$ER ( i ) = {log}_{2} {f}_{x} ( i )$$

$$nScore ( i ) = a \times ReLU ( ER )$$

Here, a is a normalization constant, and the ER (i) is scaled to 0 to 1 by

$$ReLU ( y ) = max ( 0 , y )$$

$$or$$

$$nScore = sigmoid ( ER ) .$$

In the machine learning described below, a value of the score is converted into an appropriate numerical value according to a processing method to be used. For example, in the case of COMBO, the score is converted into-1 to 0 and used for machine learning.

The actual measurement value of binding to the target is not particularly limited. It is preferable that the actual measurement value of binding to the target is measured by ELISA. The binding to the target may be an index of functions such as affinity (binding activity), target specificity, substrate specificity, and catalytic activity. The binding to the target may be an index of structural stability, thermal stability, pH stability, aggregation properties, salt stability, pressure stability, reduction stability, and modifier stability depending on the measurement conditions.

### 3. Machine Learning

In the present invention, machine learning is performed using, as training data for machine learning, scores selected based on actual measurement values of some mutants and sequence information on the mutants. That is, the artificial intelligence is caused to learn sequence information on mutants corresponding values acquired for some mutants in the library, and predicts and ranks scores of all mutants in the library. In the machine learning, for example, Bayesian optimization is preferred.

The amino acid sequence information is input by converting characters into numerical values (numerical vectors). As such a method, a method known in the field can be used, and for example, T-scale, Z-scale, ST-scale, BLOSUM, FASGAI, MSWHIM, Prot FP, ProtFP-Feature, VHSE, Aromaphilicity, and PSSM can be used (van Westen et al., J Cheminform. 2013; 5:41).

The “Bayesian optimization” is a hyperparameter tuning method, that is, one of machine learning methods for determining an optimum value (maximum value or minimum value) of a function (black box function) whose form is unknown. Each candidate point is represented by a numerical vector called a descriptor. In each iteration, a machine learning model is trained using data of the candidate points evaluated so far, and a predicted value and a predicted variance of a model function for the remaining candidate points are calculated using the trained model. A score depending on the predicted value and the predicted variance is calculated, and a candidate point having the highest score is determined as the next evaluation point to perform function evaluation. The new data obtained here is added to the training data.

In the “Bayesian optimization”, known software can be used. For example, 2DMAT (https://www.pasums.issp.u-tokyo.ac.jp/2dmat/), COMmon Bayesian Optimization Library (COMBO) (Ueno et al., Mater. Discov., 4, 18-21 (2016), https://tomoki-yamashita.github.io/CrySPY_doc/), CrySPY (https://tomoki-yamashita.github.io/CrySPY_doc/), and PHYSBO (optimization tools for PHYsics based on Bayesian Optimization) (https://www.pasums.issp.u-tokyo.ac.jp/physbo/) are known, and the known software is not limited to the examples. Among them, COMBO is preferred.

### 4. Production of Second Library

The artificial intelligence predicts and ranks score values of all mutants in the library by machine learning using data of some mutants. A library in which the desired proteins are enriched more than the initial library can be prepared by selecting suitable mutant based on the prediction result. The enriched library is referred to as a “second library” in this specification.

If necessary, the library may be enriched two times or more. That is, the second library is produced from the initial library, and then the second library is used as an initial library to produce a third library. The enrichment can be performed several times by iterating this process. The “two or more properties” used for the first enrichment may be the same as or different from properties used for the second and subsequent enrichments. After the second time, the enrichment may be performed with two or more properties, or may be performed with one property.

According to the design of the degenerate codon, the second library preferably includes a sequence that is not predicted by the machine learning. Here, the unpredicted sequence is preferably a sequence comparable to the sequence predicted by the machine learning.

### 5. Production of Optimized Protein

With function prediction through the machine learning, mutants optimized for two or more properties can be selected from the second, third, and subsequent libraries. The best mutant may be selected by actually expressing the predicted mutants and evaluating and confirming the properties thereof. In consideration of industrial use, it is generally preferable that the number of mutation sites is small. Therefore, finally, the optimum protein (mutant) is determined in consideration of the improvement in the function and the number of mutations to be introduced.

### EXAMPLES

The present invention will be specifically described below with reference to Examples, and the present invention is not limited to these Examples.

### [Example 1] Function Creation of Antibody-like Molecules

An antibody or an antibody-like molecule having a specific molecular recognition ability can be obtained by a selection operation using a genotype-phenotype integrated system such as biopanning from a molecular library based on a phage display method. However, it is often not possible to obtain mutants having appropriate desired functions and physical properties. In recent years, a next generation sequencer (NGS) is used to create indirect sequence-function association data in which a mutant having a sequence with a high enrichment rate is regarded as a highly functional mutant, and machine learning is performed to attempt to obtain a desired functional molecule. However, in many cases, specific mutants do not show appropriate enrichment during the selection operation and even training data cannot be obtained. In this Example, for the purpose of creating an antibody-like molecule, as a development of a machine learning process capable of obtaining a desired functional molecule even from a biopanning operation by which a mutant having appropriate functions and physical properties has not been obtained, training data was created by selecting an appropriate sublibrary based on NGS analysis, a second library also including a sequence not predicted by machine learning was constructed based on a sequence population predicted by machine learning, and the mutant having appropriate functions and physical properties was acquired.

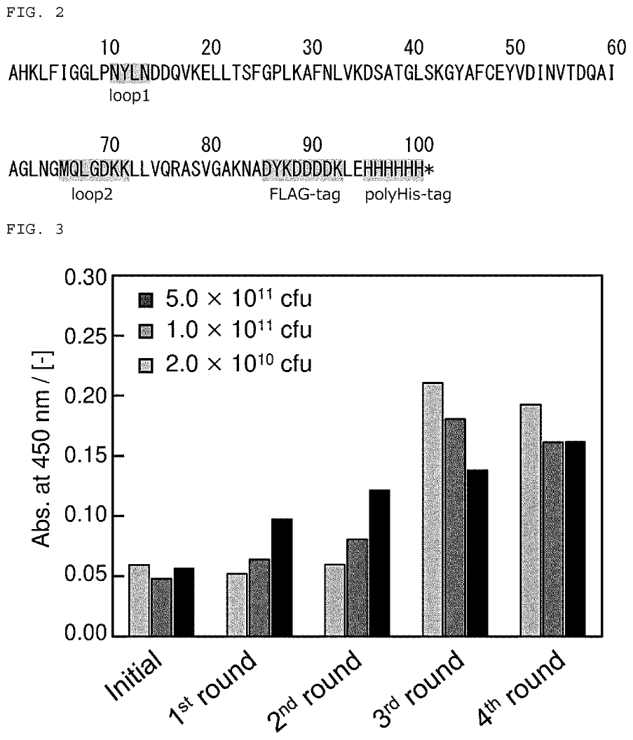

A protein obtained by substituting cysteine at the 48th location of the protein (SEQ ID NO: 1) of Protein Data Bank No. 2u2f with alanine was used as a scaffold protein of antibody-like molecules, and the mutation was performed at the residue locations in two loop regions (loop 1: 11th to 14th locations (NYLN: SEQ ID NO: 2), loop 2: locations 66th to 72nd (MQLGDKK: SEQ ID NO: 3)) of the 2u2f protein (FIG. 2). In order to give 2u2f the function of molecular recognition, a biopanning operation was performed with Galectin-3 as one of cancer markers as a target (FIG. 1B). Galectin-3 is one of the Galectin family for recognizing a sugar chain containing B-galactoside and is a molecule of interest that is not only used as a biomarker for heart failure or cancers, but also used as a new drug discovery target. For the selection operation, a phage display method with M13 phage was used. In the selection operation, first, an M13 phage library bearing a 2u2f mutant was produced. Next, a biopanning operation in which a phage bearing a mutant exhibiting target-binding properties was selected and amplified as one cycle was performed several times, and then several hundred kinds of phages were separated from the obtained phage group to obtain a phage having target-binding properties. Furthermore, the promising mutants having target-binding properties were measured for functions thereof even in a state of being separated from phages, and the availability of antibody-like molecules was evaluated.

### 1. Phage Library Preparation and Biopanning Operation

PCR was performed using a primer for randomizing the two loop regions (loop 1, 2) of 2u2f so as to have the same amino acid appearance frequency as that of CDR appearing in a human non-immune antibody library (Naïve library) (Kruziki et al., “A 45-Amino-Acid Scaffold Mined from the PDB for High-Affinity Ligand Engineering,” Chemistry & Biology, 22, 946-956 (2015)). The obtained gene fragment was inserted into a pUC vector in the form of adding a pIII protein of the M13 phage to the C-terminal. The E. coli TG-1 strain was transformed by electroporation using the obtained plasmid, and an M13 phage library of 1.0×10⁹ scale was produced using this transformant.

A biopanning operation was performed using the produced phage library (FIG. 1B). First, a selection operation of a target-binding phage was performed. In the selection operation, a negative selection of removing a phage nonspecifically adsorbed to a magnetic particle on which a target molecule was not immobilized was performed using phages of 5.0×10¹¹ cfu ((ii) in FIG. 1B), then the remaining phage solution was mixed with magnetic particles on which the target Galectin-3 was immobilized, phages not bound to the target Galectin-3 was removed by washing ((iii) FIG. 1B), and positive selection of eluting and in collecting the phages bound to the target Galectin-3 was performed to obtain a sublibrary “eluted phage” ((iv) in FIG. 1B). Next, the E. coli TG-1 strain was infected with the eluted phage and grown overnight on an agar culture medium containing ampicillin and glucose to obtain a sublibrary “infected E. coli” ((v) in FIG. 1B). The infected E. coli was cultured in a liquid culture medium and subjected to superinfection with a helper phage to produce and amplify phages, thereby obtaining a sublibrary “amplified phage” ((vi) in FIG. 1B). The above steps were iterated again using the “amplified phage” for four rounds in total.

After the selection operation, in order to evaluate whether a mutant with target-binding properties was selected, polyclonal phage ELISA was performed using an initial library and amplified phages after each round, and binding to Galectin-3 was evaluated. As a result, it was suggested that an increase in the signal was shown as the round was iterated, and mutants having affinity with the target were selected by the biopanning operation (FIG. 3).

Then, in order to obtain mutants exhibiting target-binding properties, monoclonal phages were prepared from the infected E. coli after 3rd round and 4th round using 96 deep-well plates for each of 186 mutants, and the binding evaluation by phage ELISA was performed. As a result, 52 samples of mutants exhibiting higher signals than the phages presenting wild-type 2u2f and not causing frame shift in the gene sequence were obtained. Among the 52 mutants, the C6 mutants (Table 1) appearing in a plurality of wells were prepared as proteins separated from phages.

TABLE 1

| Name | Loop 1 | Loop 2 |
|---|---|---|
| WT | NYLN (SEQ ID NO: 2) | MQLGDKK (SEQ ID NO: 3) |
| C6 | NGDG (SEQ ID NO: 4) | GYPTDSC (SEQ ID NO: 5) |

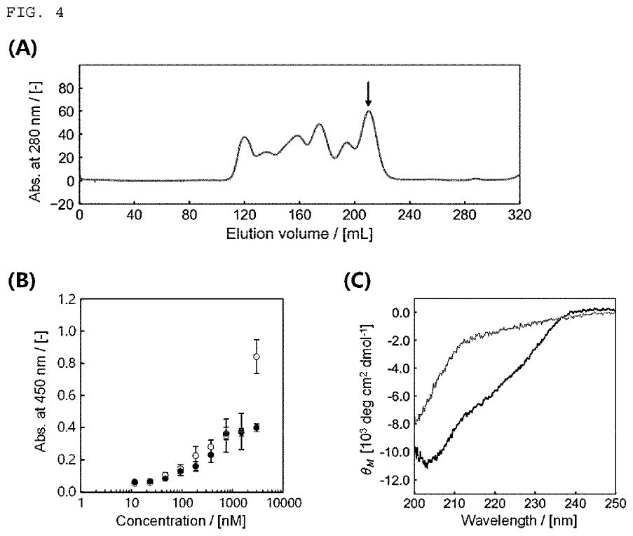

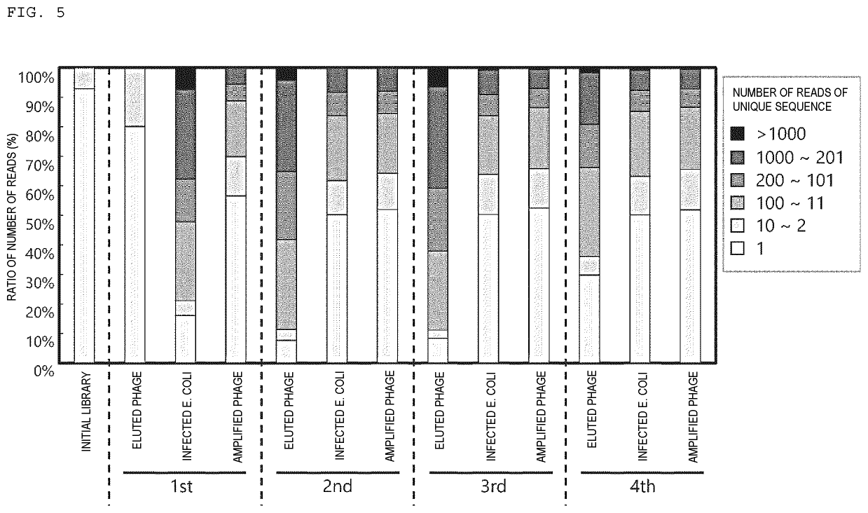

The E. coli BL21 (DE3) strain was transformed using the plasmid produced by transferring the C6 mutant gene inserted into a phagemid vector to a pET vector. After culturing, purification by immobilized metal ion affinity chromatography (IMAC) and size exclusion chromatography (SEC) was performed. As a result, unlike wild type 2u2f in a state in which a mutation was not introduced, the purified protein was expressed in various association states ((A) of FIG. 4), and when a fraction forming a monomer in the purified protein was subjected to a binding evaluation by ELISA, the fraction was bound not only to Galectin-3 as a target molecule but also to NeutrAvidin used as an anchor for immobilizing Galectin-3 on a plate, and did not have target specificity ((B) of FIG. 4). When a secondary structure of the purified protein was evaluated by a circular dichroism (CD) spectrum measurement, it was found that the secondary structure largely changed as compared with the wild-type 2u2f, and the three-dimensional structure was not maintained to be a natural structure ((C) of FIG. 4). From the above, as a result of performing a biopanning operation using 2u2f as a scaffold protein, a mutant having affinity with the target was selected. However, a target-specific mutant could not be isolated.

### 2. Next Generation Sequence (NGS) Analysis

(1) A DNA was extracted from the phage population or the E. coli population selected in the biopanning operation performed in (2) of the 1. item. The (i) to (vi) in FIG. 1B that include sublibraries such as “eluted phage”, “Infected E. coli”, and “amplified phage” in addition to the “initial phage library” were collected, and 2u2f mutant sequence fragments in the respective sublibraries were amplified by PCR and purified using agarose gel electrophoresis, followed by performing NGS analysis.

MiSeq manufactured by Illumia was used for the NGS analysis. For the analysis, 2×250 paired-end analysis for analyzing a sequence having 250 nucleotides from both the 3′ end and the 5′ end of the target DNA was used. In the nucleotide sequence data output after the analysis was ended, the nucleotide with poor analysis accuracy was removed (quality trimming), and then the nucleotide sequences analyzed from the 3′ end and the 5′ end were combined (paired-end merge). Then, sequences in the decoded data were translated from a start codon, and a sequence in which one or more residues were substituted, deleted, or inserted in a framework other than the mutated loop region was removed, and as a result, the number of read sequences in Table 2 was obtained for each sublibrary.

TABLE 2
NGS analysis result (summary of the number of reads)

| Round | Sample | Number of reads |
|---|---|---|
| | Initial phage | 318,894 |
| 1st | Negative phage | 314,902 |
| | Washed phage | 395,196 |
| | Eluted phage | 289,240 |
| | Infected E. coli | 357,094 |
| | Amplified phage | 364,228 |
| 2nd | Negative phage | 297,689 |
| | Washed phage | 389,459 |
| | Eluted phage | 313,992 |
| | Infected E. coli | 327,167 |
| | Amplified phage | 362,611 |
| 3rd | Negative phage | 309,809 |
| | Washed phage | 313,756 |
| | Eluted phage | 326,313 |
| | Infected E. coli | 360,125 |
| | Amplified phage | 311,644 |
| 4th | Negative phage | 274,021 |
| | Washed phage | 268,346 |
| | Eluted phage | 260,695 |
| | Infected E. coli | 273,863 |
| | Amplified phage | 264,167 |

In order to determine an effective sublibrary for training data for machine learning, a sequence group obtained by the NGS analysis was used to specify rounds and operations in which mutants were enriched. In the NGS analysis, the number of analyzed sequences is referred to as the number of reads, and an inherent sequence that does not overlap among the sequence group output from the NGS is referred to as a unique sequence. The larger the increase width in the number of reads of each unique sequence compared between rounds or operations is, the stronger the sequence enrichment is.

In order to observe the round and operation in which the sequence enrichment occurred, a ratio of each unique sequence in the sequences read by the NGS was calculated and compared between the sublibraries (FIG. 5). As a result, enrichment of specific mutants was observed from the amplified phages (1st round) to the eluted phages (2nd round) and then from the amplified phages (2nd round) to the eluted phages (3rd round). The comparison of these sublibraries means a direct comparison of the input to the output in each selection operation, and it is suggested that the selection operation based on the binding affinity functions well in the 2nd round and the 3rd round. However, a large enrichment of the specific mutants was observed in the stages from the eluted phages to the infected E. coli in the 1st round, and dispersion of the distribution was observed conversely in the stages from the eluted phages to the infected E. coli in each of the 2nd round, the 3rd round, and the 4th round. Therefore, it can be said that a bias other than the binding affinity to the target is applied in the E. coli infecting operation stage (v).

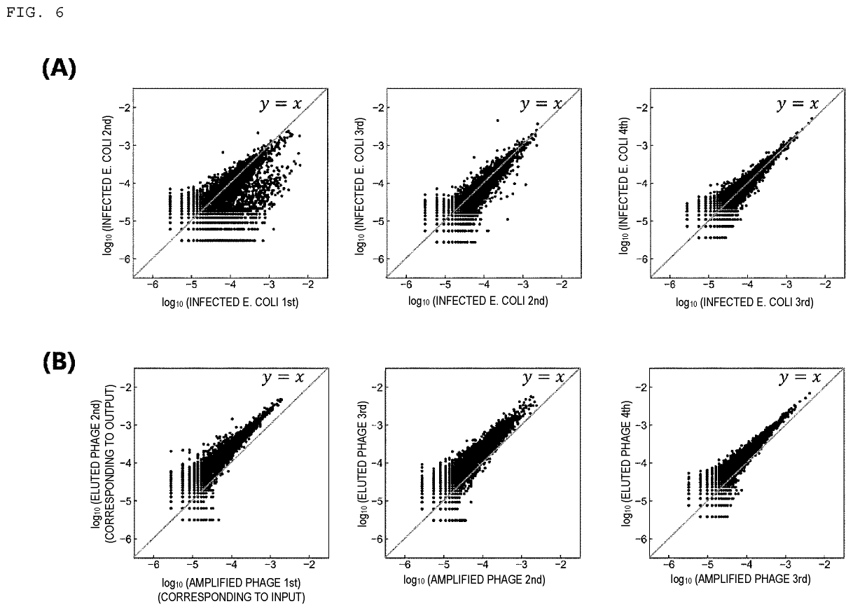

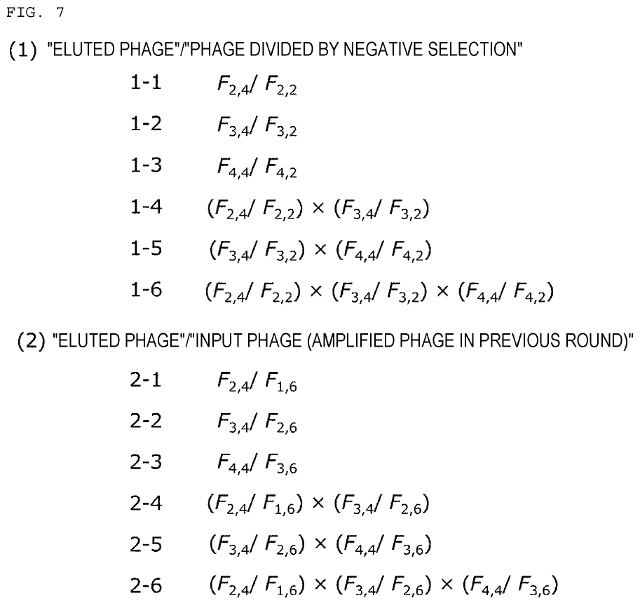

Subsequently, in order to analyze the enrichment rate of each mutant occurring in the biopanning operation, the abundance rate of each unique sequence was compared between the sublibraries. First, the abundance rate of each unique sequence in each sublibrary (the number of reads of the unique sequence/the total number of reads of the sublibrary) was calculated, and as an enrichment rate analysis between rounds, the abundance rates were compared from the 1st round to the 2nd round, from the 2nd round to the 3rd round, and from the 3rd round to the 4th round using infected E. coli sublibraries ((A) of FIG. 6). As a result, almost all mutants did not show a change in the abundance rate between rounds and were distributed in the vicinity of the straight line of y=x, and therefore, it can be said that the enrichment of mutants cannot be observed even when the outputs after the E. coli infecting operation stage are compared between rounds. On the other hand, when the abundance rates were compared in stages from the amplified phages (1st round) to the eluted phages (2nd round), stages from the amplified phages (2nd round) to the eluted phages (3rd round), stages from the amplified phages (3rd round) to the eluted phages (4th round), that is, from the input to the output in the biopanning operation in the 2nd, 3rd, 4th round, the abundance rate increased from the input to the output, and there were a large number of mutants that shifted above the y=x line ((B) of FIG. 6). Therefore, it was suggested that the enrichment of each mutant can be observed by comparing rounds using an input in the previous round and an output in the current round.

### 3. Creation of Indirect Sequence-Function Association Training Data

As a result of 2, it was found that the mutants were enriched from the amplified phages to the eluted phages in the 2nd round and the 3rd round. The enrichment in the biopanning operation indicates that more molecules are bound to the antigen than other mutants, and therefore, more enriched mutants have higher binding force than other mutants, and an increase in the abundance rate from the amplified phages to the eluted phages can be regarded as binding affinity. It can also be considered that mutants exhibiting enrichment in different rounds are more likely to bind to the target.

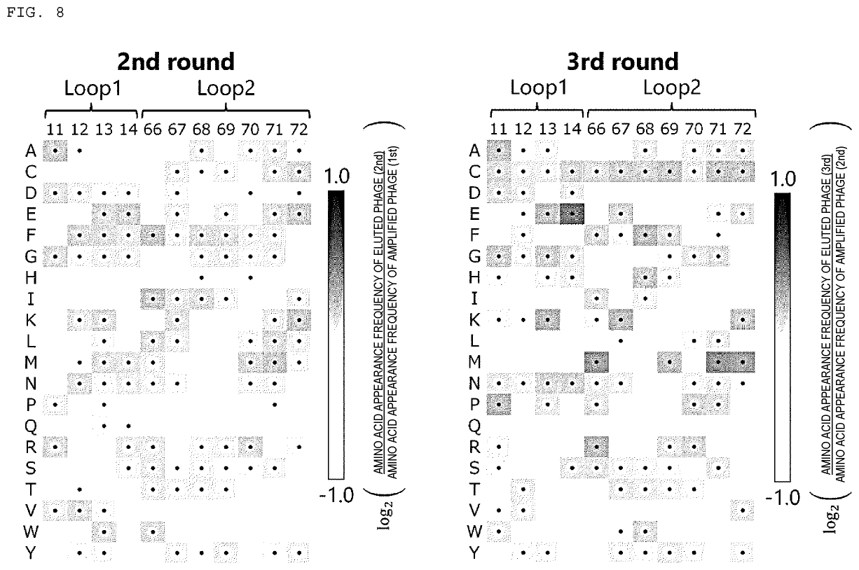

Next, among 52 samples selected from the results of the monoclonal phage ELISA of 1., 6 mutants containing C6 mutants and 11 samples determined not to bind to the target from the same results of the monoclonal phage ELISA were extracted, the results of the monoclonal phage ELISA were used to calculate score values to be associated with the sequence using the formula shown in FIG. 7, and the AUC (Area Under the Curve) values were compared with one another (Table 3) . . . . As a result, a score value calculated with eluted phages/input phages (amplified phages in the previous round) had a high AUC value, and in particular, the AUC values of the formulas 2-2, 2-4, 2-5, and 2-6 exceeded 0.7. This time, the formula 2-4 was used among the formulas whose AUC values exceeded 0.7.

TABLE 3
AUC value from score value calculated from each formula
(1) “Eluted phage”/“phage removed by negative selection”

| Formula | 1-1 | 1-2 | 1-3 | 1-4 | 1-5 | 1-6 |
|---|---|---|---|---|---|---|
| AUC value | 0.28 | 0.65 | 0.23 | 0.52 | 0.53 | 0.42 |

(2) “Eluted phage”/“input phage (amplified phage in previous round)”

| Formula | 2-1 | 2-2 | 2-3 | 2-4 | 2-5 | 2-6 |
|---|---|---|---|---|---|---|
| AUC value | 0.5 | 0.82 | 0.58 | 0.78 | 0.82 | 0.77 |

Based on the results of 2, and 3, the enrichment rate (ER (i)) of the mutant i was defined.

$$[ Math . 4 ]$$

$$ER ( i ) = {log}_{2} ( \frac{{F}_{2 , 4} ( i )}{{F}_{1 , 6} ( i )} ) + {log}_{2} ( \frac{{F}_{3 , A} ( i )}{{F}_{2 , 6} ( i )} )$$

$$nScore ( i ) = α \times ReLU ( ER )$$

F_{x, n} (i) represents an abundance rate of the mutant i in the sublibrary n. Then, a value assigned to ReLU function (ReLU(y)=max (0, y)) which is equal to 0 when ER (i) is a negative value and returns the ER (i) as it is when ER (i) is a value of 0 or more was normalized using a constant a that is set so that the highest value is 1. Using this function, normalized score values of mutants appearing in all the sublibraries including the amplified phages (1st round), the eluted phages (2nd round), the amplified phages (2nd round), and the eluted phages (3rd round) were calculated, and indirect sequence-function association data was acquired.

### 4. Production of Prediction System by Machine Learning

The above data was used as the training data, and machine learning for predicting a function evaluation value of an unknown mutant based on an amino acid sequence was performed. The prediction system was produced using COMBO which is high-speed Bayesian optimization software (op. cit., Ueno et al., 2016, etc.). The sequence data of mutants was expressed by using an index expressed by a 1 to 10 dimensional vector per residue or an appropriate combination thereof (op. cit., van Westen et al., 2013) according to the previous report.

Next, a sequence group (prediction space) whose function value is to be predicted was defined. Assuming that the number of kinds of amino acids appearing at the residue location n is represented by Ln (n=1 to 11), the scale of the prediction space can be expressed as Prediction space=L1×L2× . . . L11. The 2u2f mutant library used in this study has 11 mutation sites, and therefore, the sequence space when all 20 kinds of amino acids appear at all sites is 2.0×10¹⁴. In this study, the number of amino acids appearing at each residue location was limited, and the prediction space was designed to have a scale of about 10⁹.

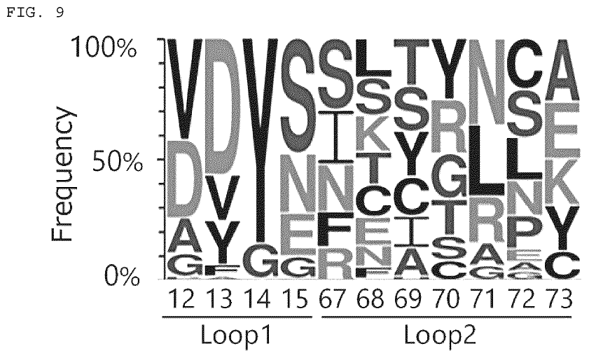

To limit the amino acid appearing in the prediction space, the enrichment rate of the amino acid at each residue location was used. The amino acid at each residue location, whose appearance frequency is increased by the biopanning operation according to 1., may be involved in binding at the location. On the other hand, the amino acid whose appearance frequency is reduced by the selection operation may not be involved in the binding or may inhibit the binding. A change rate of the amino acid appearance frequency was calculated from the amplified phages (1st round) to the eluted phages (2nd round), and from the amplified phages (2nd round) to the eluted phages (3rd round), in which the enrichment of mutants having binding affinity was suggested (FIG. 8). Here, the appearance frequency of an amino acid k at a residue location m in the sublibrary n of interest was calculated as

$$[ Math . 5 ]$$

$$Amino acid appearance frequency = \frac{\begin{matrix}Number of amino acid k appearing \\ at residue position m in sublibrary n\end{matrix}}{Number of sequences in sublibrary n} .$$

As a result of selecting the amino acid whose appearance frequency was increased in both rounds, the scale of the prediction space of the amino acids appearing at each residue location was able to be narrowed down to 9.2×10⁸ (Table 4).

TABLE 4
Amino acid residues appearing at each residue
location used in prediction space

| | Location | Used amino acids |
|---|---|---|
| Loop 1 | 12 | A D G P R V |
| | 13 | A D F G K N T V Y |
| | 14 | E G K N P Y |
| | 15 | D E G N S |
| Loop 2 | 67 | F I N R S |
| | 68 | C E F K L N S T Y |
| | 69 | A C F H I S T Y |
| | 70 | C F G R S T Y |
| | 71 | A G L N R |
| | 72 | A C E F G L N P S |
| | 73 | A C E K Y |

### 5. Narrowing Down Promising Mutants Using Prediction System

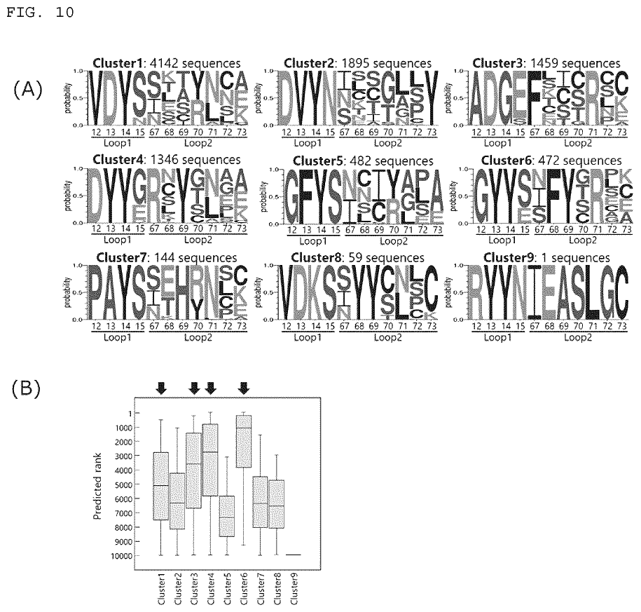

In the constructed prediction system, predicted values of all mutants included in a sequence space in which specific amino acids (Table 4) appear at 11 residue locations (11th to 14th, 66th to 72nd in FIG. 2) were calculated, and the predicted top 10,000 sequences were regarded as promising mutants (FIG. 9).

### 6. Design of Second Library

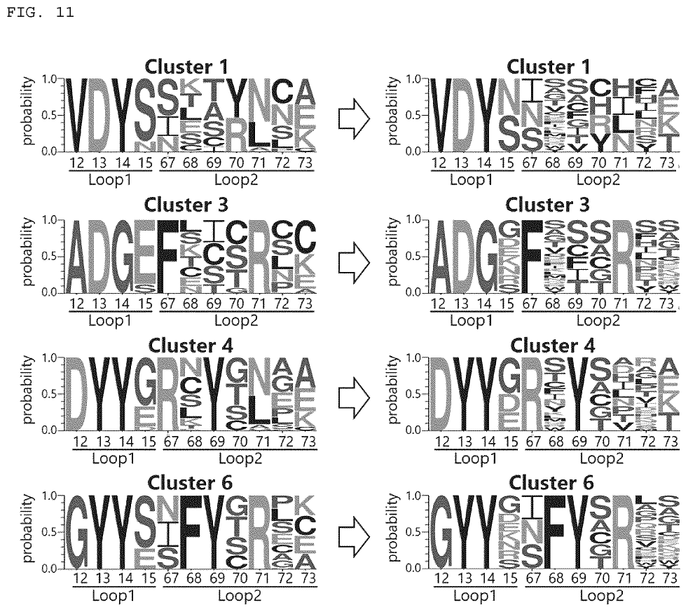

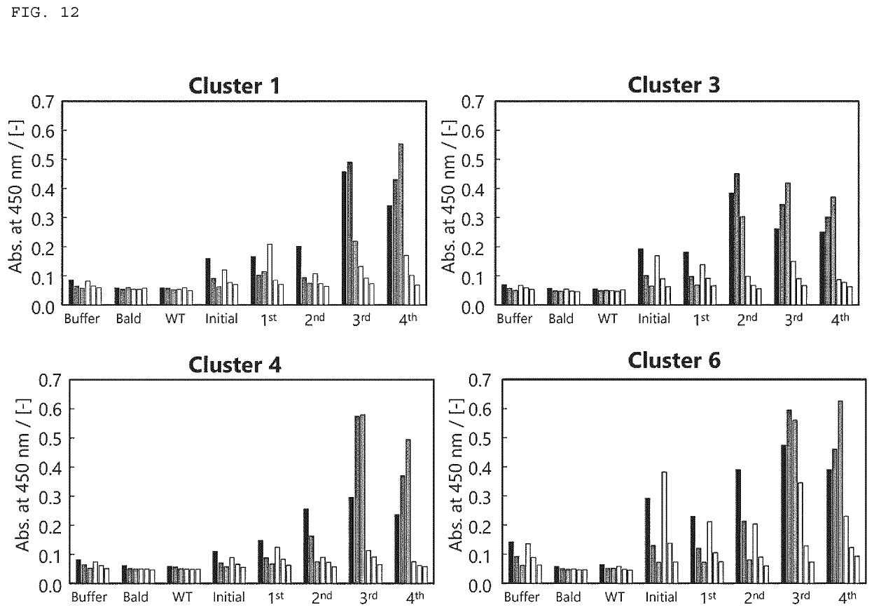

In order to prepare a second library including the top 10,000 sequences predicted by machine learning in 5, and perform biopanning using a phage display, similar sequences were grouped for the top 10,000 sequences predicted by machine learning. For the grouping, the pairwise alignment of all the top 10,000 sequences was performed using Basic Local Alignment Search Tool (BLAST) (Crooks et al., WebLogo: A sequence logo generator, Genome Research, 14, 1188-1190 (2004)), and a sequence having an e-value of 0.1 or less, which is the similarity of the sequences, was regarded as a similar sequence. At this time, the alignment was performed with settings by which any gaps are not included in the sequence. As a result, the top 10,000 sequences predicted by machine learning were roughly classified into nine clusters, and the clusters were named Clusters 1 to 9 in descending order of the number of sequences included in the cluster ((A) of FIG. 10). Then, when observing the rank distribution of the amino acid sequence included in each cluster, it was found that Clusters 1, 3, 4, and 6 among Clusters 1 to 9 included a sequence ranked into the predicted top 1,000, and a proportion of mutants having a high machine-learning prediction rank is high as a whole ((B) of FIG. 10).

Here, the design of the phage library gene group including sequences included in Clusters 1, 3, 4, and 6 including mutants having a high machine-learning prediction rank was performed using degenerate codons. In each Cluster, the appearance frequency of amino acids at each residue location was calculated based on the sequence population in the Cluster to design a degenerate codon by which a 2u2f mutant gene group in which a residue having an appearance frequency of 5% or more appears can be produced. Specifically, the amino acid caused to appear was determined, and then, codon design was performed based on the following viewpoint.

(i) Amino acids (appearance frequency of 5% or more) proposed by the prediction system must appear.

(ii) An unnecessary amino acid is not caused to appear as much as possible.

(iii) A stop codon of TAA or TGA does not appear, but the TAG stop codon is not caused to appear as much as possible.

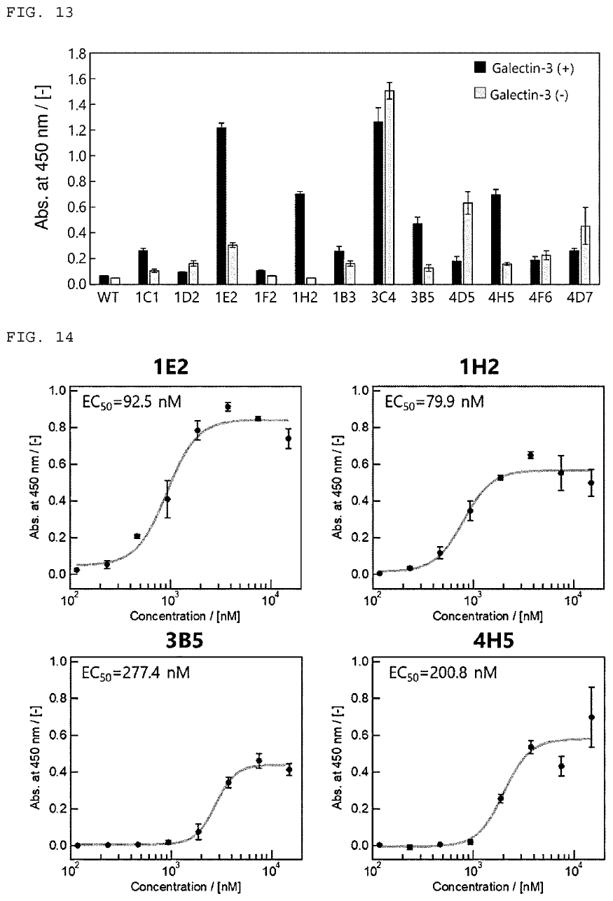

As a result, codons by which amino acids are caused to appear at each residue location, and excess amino acids were eliminated as much as possible could be designed for each cluster, and sequences not included in the machine learning prediction were also present, and the proportions of desired mutants included in the designed libraries were 0.82%, 0.33%, 1.18%, and 0.18% in Clusters 1, 3, 4, and 6, respectively (FIG. 11, Table 5). Although the proportion of the sequence predicted by machine learning is small, it is considered that there is a possibility that a mutant for which the predicted sequence is further optimized can be obtained by using a library including a sequence comparable to the predicted sequence, and an M13 phage library was prepared based on the codon design.

TABLE 5
The number of sequences of each cluster
and sequence space of designed library

| | (1) The number of | (2) Sequence space | |
|---|---|---|---|
| | sequences in cluster | of designed library | (1)/(2) [%] |
| Cluster 1 | 4.1 × 10³ | 5.0 × 10⁵ | 0.82 |
| Cluster 3 | 1.5 × 10³ | 4.5 × 10⁵ | 0.33 |
| Cluster 4 | 1.3 × 10³ | 1.1 × 10⁵ | 1.18 |
| Cluster 6 | 0.4 × 10³ | 2.2 × 10⁵ | 0.18 |

### 7. Production of Phage Library and Second Biopanning

The second library was produced using primers for which degenerate codons were designed, and an M13 phage library bearing a 2u2f mutant was prepared on a scale of 10⁸. This scale is 100 times or more the sequence space of each library, and therefore, it can be said that a phage library including not only a cluster sequence predicted by machine learning but also all mutants included in each library can be prepared.

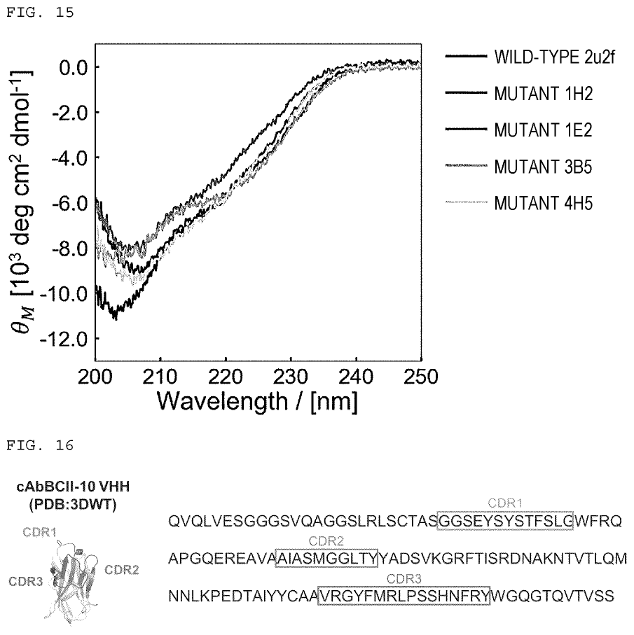

Next, when the biopanning operation was performed using the prepared second phage library and polyclonal phage ELISA was performed using the amplified phage group in each round, all clusters exhibited an increase in signals as the rounds were iterated (FIG. 12). At this time, mutants in Cluster 6, which also exhibited binding in a well on which only NeutrAvidin was immobilized, were also enriched, and polyclonal phages in the other Clusters 1, 3, and 4 exhibited specific binding.

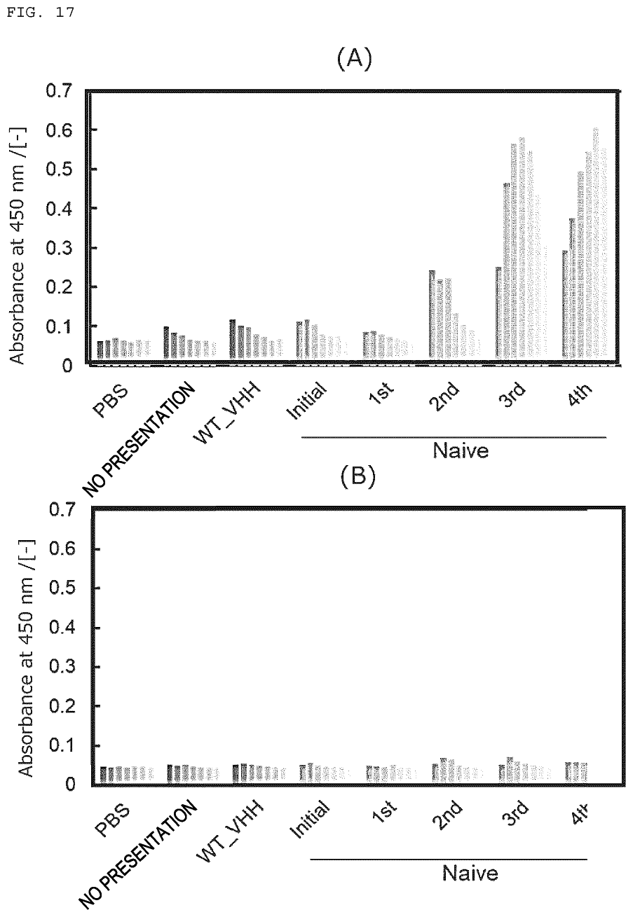

When 88 clones were isolated from the mutant group in each library after the 3rd round and screening of mutants that specifically bind to the target Galectin-3 was performed using the monoclonal phage ELISA, a total of 63 mutants exhibiting specific binding to Galectin-3 were obtained in which 20 kinds of mutants were obtained from Cluster 1, 14 kinds of mutants were obtained from Cluster 3, 20 kinds of mutants were obtained from Cluster 4, and 9 kinds of mutants were obtained from Cluster 6. Here, each mutant was named by the well number of the obtained 96-well plate, starting with the number of a cluster from which the mutant was originated. For example, a mutant obtained from Cluster 1 and cultured in the E2 well is named by “1E2”. n order to narrow down candidate molecules from the obtained 63 mutants, first, the selected mutant genes were transferred from a phagemid vector to a pET22b vector for protein expression. Then, mutants expressed in the small-scale culture using a 96-deep well plate were evaluated by Blue Native PAGE (BN-PAGE) as to whether they were expressed as monomers, and the mutants were narrowed down into 12 kinds, followed by further culturing on a scale of 500 mL and performing purification from a soluble fraction with IMAC and SEC, and 11 kinds of mutants were obtained as monomers. For the obtained mutants, whether the produced mutant exhibited binding to Galectin-3 was evaluated using ELISA, and as a result, the 1E2, 1H2, 3B5, and 4H5 mutants exhibited superior binding to Galectin-3 (FIG. 13).

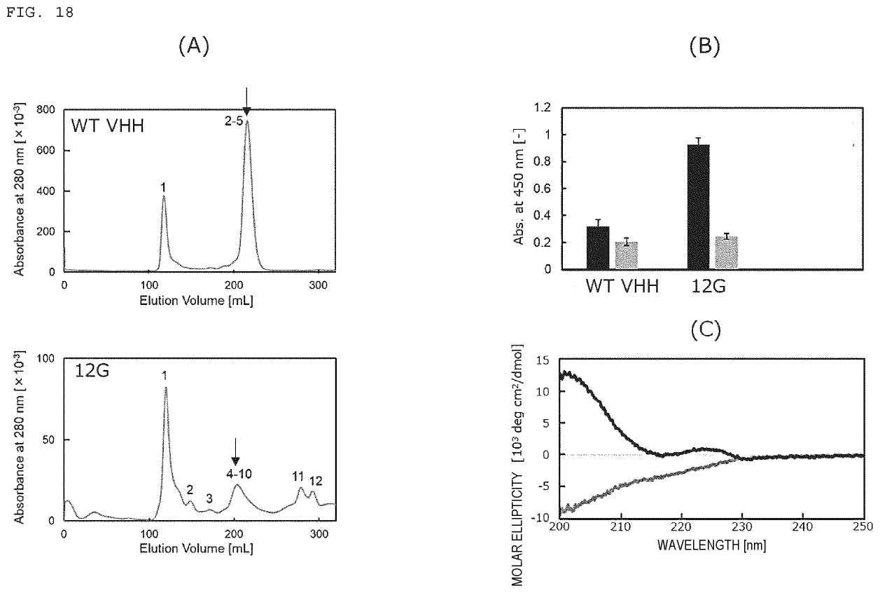

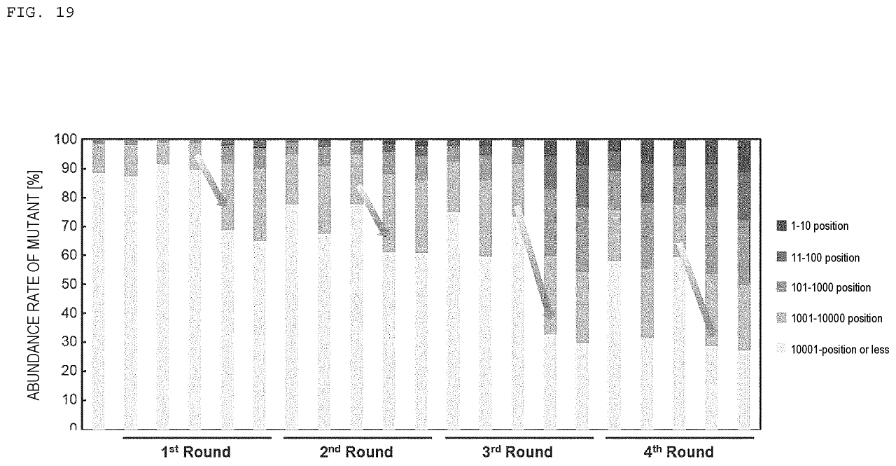

Next, regarding the four kinds of mutants exhibiting specific binding to the target Galectin-3, in order to quantify the affinity thereof, eight 2-fold dilution series were prepared starting from 1.5 μM, and an EC₅₀ value was calculated based on the binding measurement using ELISA. As a result, EC₅₀ of the 1E2, 1H2, 3B5, and 4H5 mutants were 92.5 nM, 79.9 nM, 277.4 nM, and 200.8 nM, respectively (FIG. 14). In order to evaluate whether these mutants form a secondary structure, the CD spectrum measurement was performed. As a result, it was found that the C6 mutants obtained only by wet experiments had a random coil structure ((C) of FIG. 4), while the 1H2 and 4H5 mutants obtained this time had a secondary structure similar to that of the wild-type 2u2f (FIG. 15). Accordingly, it was possible to obtain mutants whose three-dimensional structure was maintained and which exhibited specificity to the target from the second library designed using the result from the prediction system, which was not found only by the wet experiments.

The 1E2, 1H2, 3B5, and 4H5 mutants were not included in the top 10,000 predicted by machine learning, and four residues in the 1E2 mutant, three residues in the 1H2 mutant, two residues in the 3B5 mutant, and two residues in the 4H5 mutants were amino acids that did not appear in the prediction space in machine learning (Table 6, each amino acid sequence is shown in SEQ ID NO: 6 to 13). Two residues in the 3B5 mutant and one residue in the 4H5 mutant were included in the prediction space of machine learning, but did not appear in Cluster 3 and Cluster 4 after clustering. According to this result, it was possible to obtain mutants having desired functions and physical properties by causing the second library to include a sequence comparable to the top sequence predicted by machine learning.

TABLE 6
Amino acid sequences of four obtained mutants
Square box: amino acid not included in prediction
space of machine learning (Table 3)
White characters:
amino acid that does not appear in
originating machine learning prediction cluster
(left side of FIG. 11)

| Name | Amino acid sequence |
|---|---|
| 1E2 | VDYN N [symbol] C [symbol] L [symbol] |
| 1H2 | VDYN S [symbol] SR [symbol] A |
| 3B5 | ADG [symbol] F [symbol] TSR [symbol] |
| 4H5 | DYYG R [symbol] YG [symbol] A [symbol] |

### [Example 2] Improvement in Function of Weak-binding Molecule Identified Based on Biopanning Method

In a genotype-phenotype integrated system such as biopanning from a molecular library according to the phage display method, it is not always possible to obtain mutants with appropriate desired functions and physical properties. In recent years, a next generation sequencer (NGS) is used to create indirect sequence-function association data in which a mutant having a sequence with a high enrichment rate is regarded as a highly functional mutant, and machine learning is performed to attempt to obtain a desired functional molecule. However, in many cases, specific mutants do not show appropriate enrichment during the selection operation and even training data cannot be obtained. In the present example, in order to create the function of the camel heavy chain antibody heavy chain variable region fragment VHH, a machine learning process was developed in which mutants having insufficient functions and physical properties obtained by biopanning were used as parent sequences, and the functions and physical properties were improved by information processing including machine learning using NGS analysis results as training data.

### 1. Phage Library Preparation and Biopanning Operation

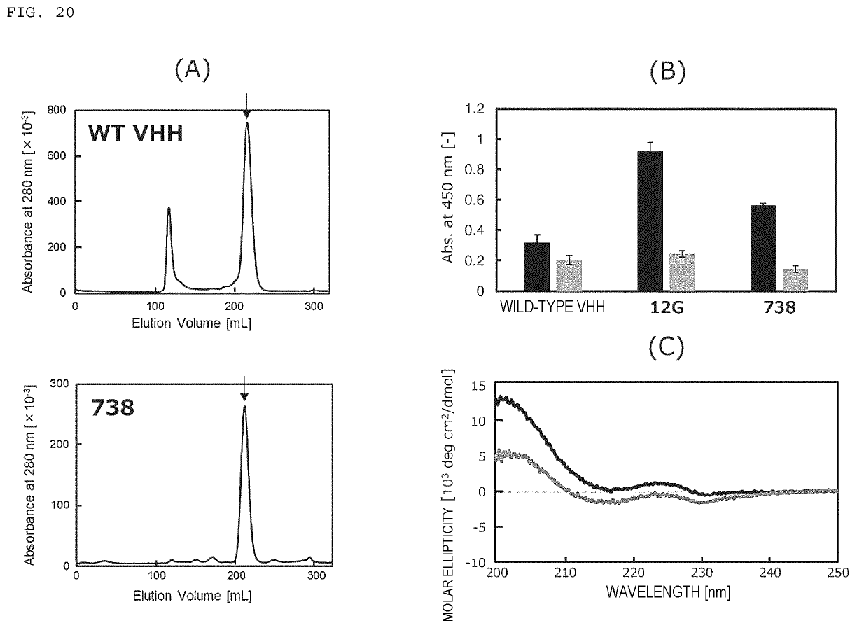

An anti-β-lactamase camel antibody fragment cAbBCII-10 VHH (PDB ID: 3DWT (SEQ ID NO: 14)) was used as a scaffold protein, and three CDRs defined by AbM were selected as mutation introduction sites (39 residues) (FIG. 16), and primers that were randomized to have the same amino acid appearance frequency as the CDRs appearing in a human non-immune antibody library (Naive library) to perform PCR in the same manner as Example 1. The obtained gene fragment was inserted into a pUC vector in the form of adding a pIII protein of the M13 phage to the C-terminal. The E. coli TG-1 strain was transformed by electroporation using the obtained plasmid, and an M13 phage library of 8.6×10⁷ scale was produced using this transformant.

The same biopanning operation as in Example 1 was performed using the produced phage library to obtain sublibraries ((i) to (vi) in FIG. 1B) such as “eluted phages”, “infected E. coli”, and “amplified phages” in the 1st round to the 4th round.

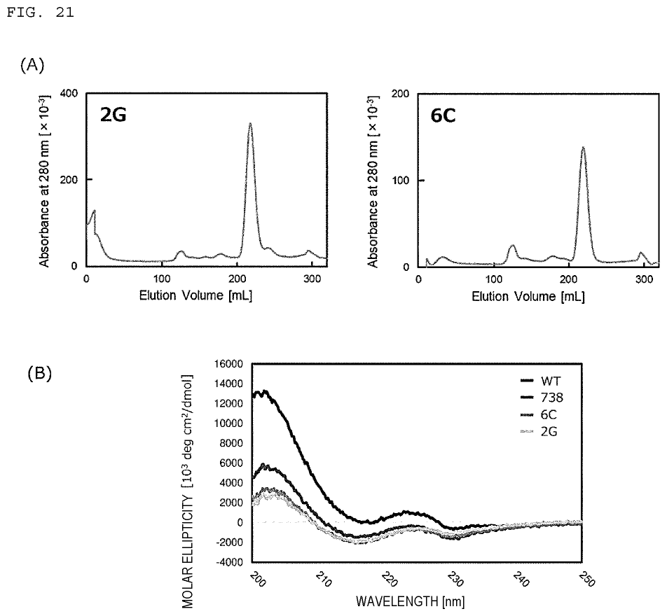

After the selection operation, in order to evaluate whether a mutant with target-binding properties was selected, polyclonal phage ELISA was performed using an initial library and amplified phages after each round, and binding to Galectin-3 was evaluated. As a result, it was suggested that an increase in the signal was shown as the round was iterated (FIG. 17), and mutants having affinity with the target were selected by the biopanning operation.

Then, in order to obtain a mutant exhibiting target-binding properties, 180 clones were isolated from the E. coli after the 4th round, a monoclonal phage was prepared using a 96 deep-well plate, and the binding evaluation by phage ELISA was performed. As a result, five mutants exhibiting signals three times or more higher than the phage bearing the wild-type VHH were obtained (7B, 11E, 11D, 4H, 12G). Then, the five mutants were attempted to be prepared as monomeric proteins separated from phages.

The E. coli BL21 (DE3) strain was transformed using a plasmid produced by transferring mutant genes inserted into phagemid vectors of five mutants exhibiting positive binding properties to a pRA5 vector. After culturing, purification by IMAC and SEC was performed. In addition, as a comparison target, two mutants (6G, 6F) showing negative binding in ELISA binding to Galectin-3 were also attempted to be prepared as a monomeric protein. As a result, only the 12G mutant was slightly eluted at the same monomer location as that of the wild-type VHH by SEC, but the yield was 1/20 or less of that of the wild-type VHH ((A) of FIG. 18). The 12G mutant prepared as a monomer exhibited specific binding properties to the target Galectin-3 in ELISA ((B) of FIG. 18), but when the secondary structure of the purified protein was evaluated by the CD spectrum measurement, it was found that the structure largely changed compared with the wild-type VHH, and the three-dimensional structure was not maintained to be a natural structure ((C) of FIG. 18).

### 2. Next Generation Sequence (NGS) Analysis

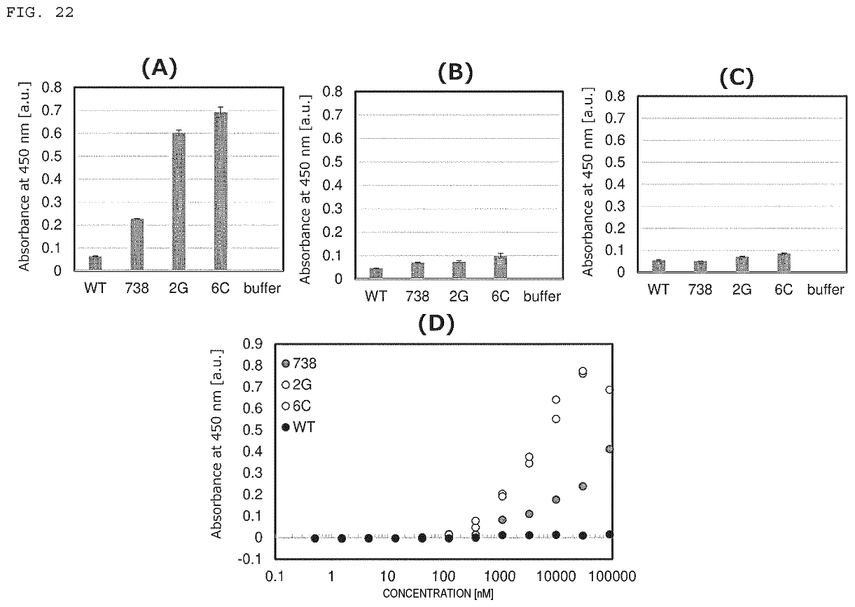

Similarly to Example 1, NGS analysis was performed on the sublibraries (i) to (vi) in FIG. 1B by using MiSeq manufactured by Illumia, and the sequence shown in Table 10 was obtained for each sublibrary. Then, in order to observe the round and the operation in which the sequence enrichment occurred in the same manner as in Example 1, a ratio of each unique sequence in the sequences read by the NGS was calculated and compared between the sublibraries (FIG. 19). As a result, similarly to Example 1, it was found that a change in distribution, which was larger than the change in distribution due to the selection operation, occurred during the E. coli infection and the amplification operation. Accordingly, it was shown that it was necessary to remove the influence of the change in distribution due to the amplification operation in the association with the function information. As a result, it was found that a change in distribution, which was larger than the change in distribution due to the selection operation, occurred from the eluted phage to the infected E. coli, and it was shown that it was necessary to remove the influence of the change in distribution due to the amplification operation in the association with the function information.

TABLE 7
NGS analysis results (summary of the number of reads)

| Round | Sample | Number of reads |
|---|---|---|
| | Initial phage | 142,273 |
| 1st | Negative phage | 137,413 |
| | Washed phage | 244,719 |
| | Eluted phage | 177,037 |
| | Infected E. coli | 280,512 |
| | Amplified phage | 253,867 |
| 2nd | Negative phage | 201,906 |
| | Washed phage | 178,435 |
| | Eluted phage | 141,884 |
| | Infected E. coli | 154,530 |
| | Amplified phage | 362,236 |
| 3rd | Negative phage | 232,800 |
| | Washed phage | 249,639 |
| | Eluted phage | 182,321 |
| | Infected E. coli | 236,358 |
| | Amplified phage | 270,365 |
| 4th | Negative phage | 310,989 |
| | Washed phage | 284,422 |
| | Eluted phage | 225,440 |
| | Infected E. coli | 142,273 |
| | Amplified phage | 137,413 |

### 3. Creation of Indirect Sequence-Function Association Training Data

Subsequently, in order to analyze the enrichment rate of each mutant occurring in the biopanning operation, score values associated with the sequence were calculated using the formulas shown in FIG. 7 using the results of the monoclonal phage ELISA for five kinds of binding-positive mutants and two kinds of negative binding mutants obtained above, and the AUC values were compared with one another (Table 8).

TABLE 8
AUC value from Score values calculated from each formula
(1) “Eluted phage”/“phage removed by negative selection”

| Formula | 1-1 | 1-2 | 1-3 | 1-4 | 1-5 | 1-6 |
|---|---|---|---|---|---|---|
| AUC value | 0.75 | 0.38 | 0.75 | 0.58 | 0.67 | 0.83 |

(2) “Eluted phage”/“input phage (amplified phage in previous round)”

| Formula | 2-1 | 2-2 | 2-3 | 2-4 | 2-5 | 2-6 |
|---|---|---|---|---|---|---|
| AUC value | 0.54 | 0.33 | 0.42 | 0.33 | 0.42 | 0.42 |

As a result, a score value calculated with eluted phages/phages removed by the negative selection had a high AUC value, and in particular, the AUC values of the formulas 1-3 and 1-6 exceeded 0.7. This time, the formula 1-3 was used among the formulas whose AUC values exceeded 0.7.

It was found that the binding-positive and binding-negative mutants can be most determined by the formula obtained by dividing the “eluted phage” in the 4th round by the “negative selection phage”.

Based on the above results, the enrichment rate (ER (i)) of the mutant i was defined.

$$[ Math . 6 ]$$

$$ER ( i ) = {log}_{2} ( \frac{{F}_{4 , 4} ( i )}{{F}_{4 , 2} ( i )} )$$

$$Score ( i ) = sigmoid ( ER )$$

4. Research for Newly Binding-positive Mutant from Mutant Group Using Clustering Analysis

When a mutant having an amino acid sequence similar to CDR of 12G was searched for from the NGS data of the mutant group after the 4th round by using the homologous sequence search program BLAST, it was possible to find 38 kinds of 12G-similar mutants by clustering analysis using a threshold value that the expected value E-value during the BLAST search was 10 or less.

Next, proteins were prepared only for mutants having a phage abundance rate of 1 or more in the “eluted phage” sublibrary in the 3rd and 4th rounds among 38 kinds of 12G-similar mutants. As a result, one similar mutant (738, Table 12) was prepared as a monomeric protein without aggregate formation ((A) of FIG. 20), and in the binding evaluation by ELISA, the 738 mutant exhibited positive binding to the target molecule ((B) of FIG. 20). Then, in the secondary structure evaluation by the CD spectrum measurement, it was found that the secondary structure similar to that of the wild-type VHH was maintained ((C) of FIG. 20).

TABLE 9
Amino acid sequence of CDR3 of 738 mutant

| Mutant | Sequence of CDR3 |
|---|---|
| 12G | PPYQHDHYIFYNINDS (SEQ ID NO: 15) |
| 738 | TNHSNEQTANHDNYIH (SEQ ID NO: 16) |

### 5. Production of Prediction System by Machine Learning

Using the training data created in the above 3., the residue location contributing to the improvement in the binding force of the binding-positive mutant 738 was predicted by machine learning. The prediction system was produced using COMBO in the same manner as in Example 1, and the sequence data of mutants was also expressed by using an index expressed by a 1 to 10 dimensional vector per residue or an appropriate combination thereof in the same manner as in Example 1.

Next, a prediction space was designed for a sequence space (₁₉C₃×20⁴=6.2×10⁸) in which mutants obtained by introducing a maximum of four residue mutations into amino acid sequences at 19 sites located in CDR3 of the 738 mutant are elements, in a sequence group (prediction space) for which a function value is to be predicted.

### 6. Design of Second Library by Prediction System

The constructed prediction system calculated predicted values of all mutants contained in the sequence space represented by the 19 residues in the CDR3. Then, four residue locations (35, 37, 38, and 39) in CDR3 in which a large number of mutations were introduced in the predicted top 1,000 sequences were determined as mutation introduction sites for the second library (Table 13).

TABLE 10
Amino acid residues appearing at each residue location used in prediction
space. Only residue location 39 contains (R) other than amino acids
that appear in 10 or more of top 10,000 sequences

| Location | Used amino acids |
|---|---|
| 35 | A D E H I K L N Q |
| 37 | I L V Y |
| 38 | K R |
| 39 | C D E G P Q G T R* |

The amino acids caused to appear at the mutation residue locations of the determined four sites were subjected to the design of the second library gene group in which the amino acids appearing in 10 sequences or more of the top 10,000 sequences predicted by the prediction system appear, using the degenerate codons, and in this case, the design was enabled only by containing the unpredicted amino acid (R) only at the residue location 39. Using primers having degenerate codons expressing the sequence space scale of 648 (9×4×2×9), PCR was performed using the 738 mutant as a template to produce the second library. The 180 clones of E. coli BL21 (DE3) transformed with a plasmid produced by inserting gene fragments of the prepared second library into a pRA5 vector were cultured on a 96 deep-well plate in a small scale, and the expressed mutants were evaluated for binding to Galectin-3 by the ELISA method. Then, two mutants specifically bound to Galectin-3 (2G, 6C) were selected, cultured on a scale of 500 mL, and purified by IMAC and SEC. As a result, it was found that both mutants could be prepared as monomers ((A) of FIG. 21), and CD spectra showed that both mutants formed secondary structures similar to that of the wild type ((B) of FIG. 21). According to the ELISA evaluation, both mutants were bound to the target Galectin-3 about 20 times more than the 738 mutant (FIG. 22).

### INDUSTRIAL APPLICABILITY

According to the present invention, an optimized protein can be efficiently obtained for a protein having a high industrial utility value, such as an antibody or an enzyme. Accordingly, modifications aimed at improving the function of the protein can be easily carried out.

All the publications, patents, and patent applications cited in the present specification are incorporated into the present specification as they are.

SEQ ID NO: 4: synthetic peptide C6 Loop 1

SEQ ID NO: 5: synthetic peptide C6 Loop 2

SEQ ID NO: 6: synthetic peptide 1E2 Loop 1

SEQ ID NO: 7: synthetic peptide 1E2 Loop 2

SEQ ID NO: 8: synthetic peptide 1H2 Loop 1

SEQ ID NO: 9: synthetic peptide 1H2 Loop 2

SEQ ID NO: 10: synthetic peptide 3B5 Loop 1

SEQ ID NO: 11: synthetic peptide 3B5 Loop 2

SEQ ID NO: 12: synthetic peptide 4H5 Loop 1

SEQ ID NO: 13: synthetic peptide 4H5 Loop 2

SEQ ID NO: 14: cAbBCII-10 VHH

SEQ ID NO: 15: CDR 3 of 12G mutant

SEQ ID NO: 16: CDR3 of 738 mutant

1. A method for producing a nucleic acid library, the method comprising:
preparing, by a phage display method, a first library composed of mutants obtained by randomly introducing a mutation into a nucleic acid sequence encoding a protein bound to or configured to be bound to a target;
biopanning the first library to obtain data to be used for machine learning from an obtained sublibrary; and
performing machine learning with the data to be used for machine learning to obtain the nucleic acid library from the first library based on a machine learning prediction,
wherein the data to be used for machine learning includes a sequence of a mutant population included in a sublibrary at a target-binding sequence elution stage, an estimated binding strength to the target, and an actual measurement value of binding of some mutants included in the mutant population to the target.

2. The method of claim 1, wherein the data to be used for machine learning is produced by the method comprising;
obtaining data of sequences and appearance frequencies of the sequences for the sublibrary at the target-binding sequence elution stage and sublibraries at one or more stages different from the stage;
calculating, based on the appearance frequencies, a score indicating the estimated binding strength to the target; and
determining, as the data to be used for machine learning, the score, the actual measurement value of binding to the target, and sequence data providing the score and the actual measurement value.

3. The method of claim 2, wherein the one or more stages are stages independently selected from the group consisting of a non-specific binding sequence removal stage, a target-binding sequence selection stage, an E. coli infecting operation stage, and a selected sequence amplification stage.

4. The method of claim 2, wherein the score is calculated using a ratio of an appearance frequency between the sublibrary at the target-binding sequence elution stage and a sublibrary at a non-specific binding sequence removal stage or a selected sequence amplification stage.

5. The method of claim 2, wherein the score is calculated by a ratio of an appearance frequency in the sublibrary at the target-binding sequence elution stage to an appearance frequency in a sublibrary at a non-specific binding sequence removal stage in the same round, or calculated by a ratio of an appearance frequency in the sublibrary at the target-binding sequence elution stage to an appearance frequency in a sublibrary at a selected sequence amplification stage in different rounds.

6. The method of claim 2, wherein the score is calculated with data of sublibraries at the 2nd to 4th round.

7. The method of claim 2, wherein the score is calculated according to any one formula selected from the following formulas 1) to 6):
$${f}_{x} ( i ) = \frac{{F}_{x , 4} ( i )}{{F}_{x , 2} ( i )} \qquad 1 )$$
$${f}_{x} ( i ) = \frac{{F}_{x - 1 , 4} ( i )}{{F}_{x - 1 , 2} ( i )} \times \frac{{F}_{x , 4} ( i )}{{F}_{x , 2} ( i )} \qquad 2 )$$
$${f}_{x} ( i ) = \frac{{F}_{x - 1 , 4} ( i )}{{F}_{x - 1 , 2} ( i )} \times \frac{{F}_{x , 4} ( i )}{{F}_{x , 2} ( i )} \times \frac{{F}_{x + 1 , 4} ( i )}{{F}_{x + 1 , 2} ( i )} \qquad 3 )$$
$${f}_{x} ( i ) = \frac{{F}_{x , 4} ( i )}{{F}_{x - 1 , 6} ( i )} \qquad 4 )$$
$${f}_{x} ( i ) = \frac{{F}_{x - 1 , 4} ( i )}{{F}_{x - 2 , 6} ( i )} \times \frac{{F}_{x , 4} ( i )}{{F}_{x - 1 , 6} ( i )} \qquad 5 )$$
$${f}_{x} ( i ) = \frac{{F}_{x - 1 , 4} ( i )}{{F}_{x - 2 , 6} ( i )} \times \frac{{F}_{x , 4} ( i )}{{F}_{x - 1 , 6} ( i )} \times \frac{{F}_{x + 1 , 4} ( i )}{{F}_{x , 6} ( i )} . \qquad 6 )$$
wherein F_{x, n}(i) is an abundance rate of a mutant i in the x-th round in a sublibrary n, the abundance rate defined by a ratio of a number of reads of unique sequence/total number of reads of sublibrary, and
n is as follows:
n=1: first library;
n=2: sublibrary from phages removed by non-specific binding phage removal;
n=3: sublibrary from phages removed at target-binding sequence elution stage;
n=4: sublibrary from phages after target-binding sequence elution stage;
n=5: sublibrary from E. coli after being infected with phages; and
n=6: sublibrary from phages after amplification.

8. The method of claim 1, wherein the actual measurement value of binding to the target is measured by ELISA.

9. The method of claim 1, wherein in the performing machine learning, the nucleic acid library includes a sequence not predicted by machine learning, depending on a design of a degenerate codon.

10. The method of claim 1, wherein the protein bound to the target or configured to be bound to the target is an antibody, an antibody-like molecule, or an enzyme.

11. A method for producing an optimized protein, the method comprising:
producing a nucleic acid library by the method of claim 1;
screening the nucleic acid library to determine a nucleic acid sequence encoding an optimized protein; and
producing a protein optimized based on the nucleic acid sequence.
